SEQUENCE LISTING (6 sequences)

SEQ ID NO: 1 - DNA, 20 bp, Artificial Sequence
  Arg1 forward primer
cagaagaatggaagagtcag

SEQ ID NO: 2 - DNA, 24 bp, Artificial Sequence
  Arg1 reverse primer
cagatatgcaggcagggagtcacc

SEQ ID NO: 3 - DNA, 26 bp, Artificial Sequence
  Fizz1 forward primer
ggtcccagtgcatatggatgagacca

SEQ ID NO: 4 - DNA, 26 bp, Artificial Sequence
  Fizz1 reverse primer
cacctcttcactcgagggacagttgg

SEQ ID NO: 5 - DNA, 21 bp, Artificial Sequence
  iNOS2 forward primer
ccgaagcaaacatcacattca

SEQ ID NO: 6 - DNA, 19 bp, Artificial Sequence
  iNOS2 reverse primer
ggtctaaaggctccgggct

(19) United States
(12) Patent Application Publication
(10) Pub. No.: US 2021/0260008 A1
(43) Pub. Date: Aug. 26, 2021

(54) COMBINATION THERAPY FOR TREATING CANCER

(71) Applicant: THE JOHNS HOPKINS UNIVERSITY, Baltimore, MD (US)

(72) Inventors: Cynthia Zahnow, Bel Air, MD (US); Robert A. Casero, White Hall, MD (US); Meghan Travers, Baltimore, MD (US)

(21) Appl. No.: 17/059,878

(22) PCT Filed: May 30, 2019
(86) PCT No.: PCT/US2019/034687

§ 371 Date: Nov. 30, 2020

Related U.S. Application Data
(60) Provisional application No. 62/677,936, filed on May 30, 2018.

(51) Int. Cl. A61K 31/198; A61K 31/706; A61K 31/16; A61K 31/4406; A61K 31/27; C07K 16/28; A61P 35/00

(52) U.S. Cl. CPC A61K 31/198; A61K 31/706; A61K 31/16; A61K 31/4406; A61P 35/00; C07K 16/2818; C07K 16/2827; C07K 16/2866; A61K 31/27

(57) ABSTRACT

The present invention relates to the field of cancer. More specifically, the present invention provides compositions and methods useful for treating solid tumors. In a specific embodiment, a method for treating a solid tumor in a patient having cancer comprises the step of administering to the patient a polyamine reduction therapy and an epigenetic therapy.

### CROSS-REFERENCE TO RELATED APPLICATIONS

This application claims the benefit of U.S. Provisional Application No. 62/677,936, filed May 30, 2018, which is incorporated herein by reference in its entirety.

### FIELD OF THE INVENTION

The present invention relates to the field of cancer. More specifically, the present invention provides compositions and methods useful for treating cancer.

### INCORPORATION-BY-REFERENCE OF MATERIAL SUBMITTED ELECTRONICALLY

This application contains a sequence listing. It has been submitted electronically via EFS-Web as an ASCII text file entitled “P14682-01_ST25.txt.” The sequence listing is 1,387 bytes in size, and was created on May 30, 2019. It is hereby incorporated by reference in its entirety.

### BACKGROUND OF THE INVENTION

Cancers are now recognized as being driven by widespread changes in the epigenome including changes in DNA methylation and chromatin packaging. Epigenetic therapies include DNA methyl transferase (DNMT) inhibitors (DNMTi), histone deacetylase inhibitors (HDACi), histone methyltransferase inhibitors and histone demethylase inhibitors. 5-azacytidine (AZA) is a demethylating agent that incorporates into nucleic acids as a cytidine analog which cannot be methylated by DNMTs. AZA is FDA approved for myelodysplastic syndrome (MDS), and low nanomolar doses lead to decreased DNA promoter methylation and restored expression of hypermethylated genes in cancer (20). Additionally, AZA treatment induces the re-expression of hypermethylated, silenced endogenous retroviruses (ERVs) in vitro, which can elicit an anti-viral, interferon immune response that leads to T cell activation in vivo (15,18). Furthermore, AZA treatment of an ovarian cancer mouse model leads to increased immune cells in the tumor microenvironment, and combination AZA and HDACi sensitized tumors to α-PD-1 therapy (18). While first generation HDACi combined with DNMTi have demonstrated some promise in clinical trials for non-small cell lung cancer (21), there remains a need to discover novel treatment strategies that activate the immune system and provide long term remission for other solid tumors.

### SUMMARY OF THE INVENTION

The present invention is based, at least in part, on the discovery that combined epigenetic and polyamine-reducing therapy stimulates M1 macrophage polarization in the tumor microenvironment of an ovarian cancer mouse model, resulting in decreased tumor burden and prolonged survival.

Thus, in one aspect, the present invention provides methods for treating cancer. In particular embodiments, the present invention provides methods for treating solid tumors in patients. Any solid tumor is contemplated including, but not limited to, ovarian, breast, melanoma, lung (e.g., small cell lung cancer (SCLC)), colon, pancreas, liver, esophageal, stomach, epithelial, sarcoma, cervical, and uterine. In certain embodiments, the cancer is ovarian, breast, SCLC or melanoma. The methods comprise a combination therapy comprising administration of a polyamine reduction therapy and an epigenetic therapy. In certain embodiments the polyamine reduction therapy comprises a polyamine synthesis inhibitor and/or a polyamine transport inhibitor. In a specific embodiment, the polyamine synthesis inhibitor comprises an ornithine decarboxylase (ODC) inhibitor. In certain embodiments, the epigenetic therapy comprises DNMTi, an HDACi, a histone methyltransferase inhibitor and/or a histone demethylase inhibitor. In particular embodiments, the combination therapy further comprises a checkpoint inhibitor.

In certain embodiments, the methods may further comprise priming the patient with a prior administration of an epigenetic therapy, and then administering the combination therapy. In further embodiments, the methods and compositions may further comprise an agent that reduces macrophages. In one embodiment, the agent is anti-CSF1R. In a specific embodiment, the agent reduces or inhibits M2 macrophages. In more specific embodiments, the agent is an anti-IL-10R antibody. In other embodiments, the agent is Tasquinimod.

In certain embodiments, the combination therapy comprises a polyamine reduction therapy and an epigenetic therapy, wherein the combination therapy does not include a checkpoint inhibitor. In a specific embodiment, the combination therapy comprises an ODC inhibitor and/or a polyamine transport inhibitor, and a DNMTi and/or an HDACi, wherein the combination therapy does not include a checkpoint inhibitor. In such embodiments, the combination can also comprise an agent that reduces M2 macrophages.

In particular embodiments, the ODC inhibitor comprises difluoromethylornithine or or other agents that downregulate polyamine biosynthesis including polyamine analogues. Alpha-difluoromethylomithine (DFMO; eflornithine) is described in, for example, U.S. Pat. Nos. 6,730,809; 6,573,290; 6,258,845; and 4,925,835. The agents that down regulate polyamine biosynthesis can include polyamine analogues including, but not limited to, N1, N11-bis(ethyl)norspermine (BENSpm) and N¹,N¹²-bis(ethyl)-cis-6,7-dehydrospermine tetrahydrochloride (PG-11047). Ornithine decarboxylase inhibitors are known and described in, for example, U.S. Pat. Nos. 5,753,714; 5,132,293; 5,002,879; 4,720,489; and 4,499,072. Examples include, but are not limited to, alpha-difluoromethylornithine, 2-(difluoromethyl)-2,5-diaminopentanoic acid; alpha-ethynyl ornithine; 6-heptyne-2,5-diamine; 2-methyl-6-heptyne diamine; alpha.-difluoromethyl ornithine; the methyl ester of monofluoromethyl dehydroornithine; the R,R-isomer of methyl acetylenic putrescine, 3-aminooxy-1-aminopropane (APA) and its analogs or derivatives such as CGP 52622A and CGP 54169A, 1,25-dihydroxycholecalciferol, and pharmaceutically acceptable salts and prodrugs thereof.

In other embodiments, the polyamine transport inhibitor comprises AMXT-1501.

In certain embodiments, the DNMTi comprises, but is not limited to, 5-azacytidine (AZA), 5-azadeoxycytidine (DAC) SGI-110 (guadecitabine) or analogs of the foregoing. In a specific embodiment, the demethylating agent comprises AZA.

In other embodiments, the HDACi comprises, but is not limited to, givinostat, entinostat or analogs thereof.

In further embodiments, the checkpoint inhibitor comprises, but is not limited to, an anti-PD1 antibody (e.g., nivolumab, pembrolizumab (keytruda)), an anti-PDL-1 antibody (e.g., Medi4736) or an anti-CTLA4 antibody (e.g., tremelimumab).

In yet another embodiment, a method for treating a solid tumor in a patient comprises the step of administering (a) a polyamine reduction therapy comprising either (i) DFMO or (ii) AMXT-1501; and (b) an epigenetic therapy comprising either (i) AZA or DAC or (ii) givinostat or entinostat. In a further embodiment, the method comprises administering (c) a checkpoint inhibitor comprising nivolumab, pembrolizumab, Medi4736, MPDL3280A or tremelimumab. In another embodiment, the method comprises administering (d) an agent that reduces M2 macrophage comprising anti-IL10R antibody.

In another aspect, the present invention provides pharmaceutical compositions. In one embodiment, a pharmaceutical composition comprises (a) either an ODC inhibitor or a polyamine transport inhibitor; and (b) either a DNMTi or a HDACi. In another embodiment, the ODC inhibitor comprises DFMO. In yet another embodiment, the polyamine transport inhibitor comprises AMXT-1501. In a specific embodiment, the DNMTi comprises AZA or DAC. In another specific embodiment, the HDACi comprises givinostat or entinostat. In a further embodiment, the composition comprises a (c) a checkpoint inhibitor. In certain embodiments, the checkpoint inhibitor comprises nivolumab, pembrolizumab Medi4736 or tremelimumab. The pharmaceutical composition can further comprise an agent that reduces M2 macrophages. In a specific embodiment, the agent comprises an anti-IL-10R antibody.

In yet another aspect, the present invention provides kits useful for treating cancer. In a specific embodiment, a kit comprises a polyamine reduction therapy and an epigenetic therapy. In a more specific embodiment, the polyamine reduction therapy comprises a polyamine synthesis inhibitor or a polyamine transport inhibitor. In another specific embodiment, the epigenetic therapy comprises a DNMTi or an HDACi. The kit can further comprise a checkpoint inhibitor and/or an agent that reduces M2 macrophages. In particular embodiments, the kit comprises instructions for administration to patients to treat solid tumors.

### BRIEF DESCRIPTION OF THE FIGURES

FIG. 1A-1E. Combination AZA+DFMO reduces tumor burden and increases survival in an ovarian cancer mouse model. FIG. 1A: Tumor cell injection and treatment schematic. Mice were injected i.p. with 250,000 VEGF-DEFB ID8 MOSE cells (VDID8). 0.5 mg/kg of AZA was given i.p. 5 days a week, every other week. 2% DFMO was provided in water bottles. Mice were treated throughout the duration of the experiment. Upon 25-30% weight gain, ascites fluid was drained from mice and processed for analysis of the tumor microenvironment. FIG. 1B: Tumor burden, represented by ascites volume, 5 weeks post tumor injection. Data is from the second ascites drain procedure, the first was 4 weeks post tumor injection. Representative data (mean +/−SEM shown). n=10; four biological replicates. Data were tested for a Gaussian distribution using Shapiro-Wilk test and found not to be normal. Significance was determined using Kruskal Wallis test; statistical outliers removed using Peirce's criterion. FIG. 1C: Representative survival curve (median survival in days); n=10; four biological replicates. Significance determined using log-rank Mantel-Cox test. FIG. 1D: Total lymphocyte populations in week 5 bulk ascites fluid of mice; n=14-21. Data were tested for a Gaussian distribution using Shapiro-Wilk test and found to be normal after log transformation. Significance was determined using one way ANOVA. FIG. 1E: Flow cytometry plots of SSC vs. FSC demonstrating an increase in lymphocyte populations in ascites fluid at week 5 post tumor injection with AZA, DFMO, and AZA+DFMO treatment. Range of total lymphocyte population percentages are included in the upper left hand corner for each plot. *p<0.05; **p<0.01; ***p<0.001; ****p<0.0001.

FIG. 2A-2G. Combination AZA+DFMO elevates lymphocyte populations and IFNγ+ lymphocytes in tumor associated ascites. Ascites fluid was collected from treated mice and the cellular fraction was processed for FACS analysis. FACs analysis of cellular populations isolated from ascites at week 5 post injection demonstrate that combination treatment of 0.5mg/kg AZA and 2% DFMO was the most effective at significantly elevating total T cells (FIG. 2A), NK Cells (FIG. 2B), and CD4+ T cells (FIG. 2C). An upward trend in total CD8+ T cells (FIG. 2D) was observed as well. Both CD4+ and CD8+ T cells (FIG. 2E, 2F) and NK cells (FIG. 2G) showed an increase in IFNγ+ cells with combination treatment. IFNγ+ NK cells were significantly increased with combination AZA+DFMO compared to both single agent AZA or DFMO alone. Each data point represents cells harvested from 1 mouse. n=14-21. All data were tested for a Gaussian distribution using Shapiro-Wilk test. Significance was determined using one way ANOVA (FIG. 2A-2B, 2E-2G) or Kruskal Wallis test (FIG. 2C-2D) dependent upon normality results from Shapiro-Wilk test.

FIG. 3A-3E. AZA-DFMO combination therapy decreases macrophages and alters the ratio of MHC II high to MHC II low macrophages. Ascites fluid was collected from treated mice and the cellular fraction was processed for FACS analysis. No changes were observed between any of the treatment arms for non-lymphocytes (FIG. 3A) or MDSCs (FIG. 3B). A significant decrease in total macrophages was observed in combination treated mice compared to vehicle, as well as a significant decrease compared to AZA alone (FIG. 3C). Further analysis of macrophage populations revealed that the MHCII low (M2-like) population was decreased in all treatment arms (FIG. 3D), while the MHCII Hi (M1-like) population was increased across treatment arms (FIG. 3E). All data were tested for a Gaussian distribution using Shapiro-Wilk test. Significance was determined using one way ANOVA (FIG. 3B-3E) or Kruskal Wallis test (FIG. 3A) dependent upon normality results from Shapiro-Wilk test.

FIG. 4A-4F. AZA+DFMO treatment reduces M2 polarization and increases M1 polarized macrophages in the tumor microenvironment. FIG. 4A: Percentage of M1 macrophages (MHCII+ CD206−) were increased with DFMO and AZA treatment, and further increased with combination AZA+DFMO treatment. FIG. 4B: Percentage of M2 macrophages (MHC II− CD206+) were reduced in all treatment arms, with the greatest reduction observed in combination AZA+DFMO treatment. Macrophages were sorted from bulk ascites fluid collected from mice at week 5 post tumor injection. qRT-PCR for Arg1 (FIG. 4C) and Fizz1 (FIG. 4D) was performed on sorted macrophages (M2 macrophages=CD45+ L/D− F4\80+ CD11b+ MHC II− CD206+; M1 macrophages=CD45+ L/D− F4\80+ CD11b+ MHC II+ CD206−). Data confirms that MHC II− CD206+ macrophages exhibited gene expression signatures typical of M2 polarization. FIG. 4E: qRT-PCR of iNOS2 in M1 macrophages vs. M2 macrophages confirming that MHC II+ CD206− macrophages exhibited gene expression signatures typical of M1 polarization. FIG. 4F: Percentage of M2 macrophages (MHCII− CD206+) increase with tumor burden in vehicle treated mice. Drain 1 was performed at week 4 post tumor cell injection; drain 2 at week 5 and drain 3 at week 6. All data were tested for a Gaussian distribution and found to be normal using Shapiro-Wilk test. Significance was determined using one way ANOVA (FIG. 4A-4B) or t test (FIG. 4C-4E).

FIG. 5A-5H. Increased M1 macrophages are essential to the efficacy of combined AZA+DFMO treatment in an ovarian cancer mouse model. FIG. 5A: Treatment schematic for dosing with macrophage block antibody α-CSF1R. All mice are drained during each ascites draining procedure beginning at week 7. FIG. 5B: Reduction in total macrophages observed at the first drain (week 7 on schematic). FIG. 5C: ELISA of CSF-1 levels demonstrating an increase in circulating CSF-1 in the presence of receptor block CSF1R. FIG. 5D: Tumor burden represented by ascites volume in mice treated with AZA+DFMO in presence of CSF1R antibody or IgG control during the second drain (week 8 on schematic in a). FIG. 5E: Tumor burden during the third drain (week 9 on schematic in a) demonstrating an increase in tumor burden in AZA+DFMO mice receiving CSF1R. FIG. 5F: Survival curve of AZA+DFMO treated mice receiving CSF1R antibody. Mice with decreased macrophages due to the antibody demonstrated a decrease in survival compared to AZA+DFMO mice receiving IgG. FIG. 5G: M1 macrophages (MHC II+ CD206−) analyzed via flow cytometry. AZA+DFMO treated mice receiving CSF1R show no increase in M1 macrophages. FIG. 5H: M2 macrophages (MHC II− CD206+) analyzed via flow cytometry. M2 macrophages were reduced in both AZA+DFMO treatment arms, compared to mock treated mice. All data were tested for a Gaussian distribution and found to be normal using Shapiro-Wilk test. Significance was determined using a t test (FIG. 5B-5C) or one way ANOVA (FIG. 5D-5E, 5G-5H).

FIG. 6A-6B. DFMO treatment of VDID8 cells in vitro reduces putrescene and spermidine levels. FIG. 6A: VDID8 cells were cultured in 10% FBS RPMI+gentamicin for one week prior to beginning 10 day treatment. Cells were treated with 500 nM AZA/saline for 10 days and 5 mM DFMO/water for 3 days. AZA+DFMO cells were treated with 500 nM AZA for the first 7 days, and 500 nM AZA + 5 mM DFMO for the final 3 days of treatment. High-performance liquid chromatography (HPLC) analysis of polyamine levels in cultured, treated VDID8 cells are shown. n=3. FIG. 6B: Bulk ascites fluid was collected from individual mice treated with DFMO, AZA, or AZA+DFMO. Bulk ascites cells were lysed and washed and remaining cells were prepped for HPLC analysis of polyamine levels. n=4-10. All data were tested for a Gaussian distribution and found to be normal using Shapiro-Wilk test. Significance was determined using a one way ANOVA.

FIG. 7A-7H. Addition of α-PD-1 therapy to AZA+DFMO treatment regimen did not further decrease tumor burden or increase survival in VDID8 ovarian cancer mouse model. FIG. 7A: Mouse treatment protocol for PD-1 experiment. Due to the size of this experiment (80 mice), experiment performed only once. FIG. 7B: Ascites fluid collected from treated mice at week 5.5; no difference in tumor burden was observed with the addition of α-PD-1. Mice survival was not extended with the addition of α-PD-1 to vehicle (FIG. 7C), alt. AZA (FIG. 7D), or 2% DFMO (FIG. 7E) treated mice. The addition of α-PD-1 to combination treated mice in fact decreased their overall survival (FIG. 7F); however only by a median survival difference of 2 days. PD-1+ CD4+ (FIG. 7G) and CD8+ (FIG. 7H) T cells analyzed via FACS showing no significant difference among treatment arms. n=10. All data were tested for a Gaussian distribution. Significance was determined using one way ANOVA (FIG. 7G-7H) or KruskalWallis test (FIG. 7B) dependent upon normality results from Shapiro-Wilk test. Note: “Alt.” refers to “alternating”; AZA doses are given alternating weeks as shown in (FIG. 7A).

FIG. 8A-8B. AZA+DFMO treatment decreases peritoneal macrophages in the tumor microenvironment. FIG. 8A: Representative flow cytometry plots demonstrating the decrease in F4\80+ CD11b+ macrophages in mice treated with AZA, DFMO, and AZA+DFMO. FIG. 8B: Within F4\80+ CD11b+ macrophage population, representative flow cytometry plots demonstrating the increase in MHCII+ macrophages in mice treated with DFMO, AZA, and AZA+DFMO.

FIG. 9. AZA+DFMO treatment increases M1 polarized macrophages and decreases M2 polarized macrophages in the tumor microenvironment. Within F4\80+ CD11b+ macrophage population, representative flow cytometry plots demonstrating the increase in M1 macrophages (CD206−MHCII+) in mice treated with DFMO, AZA, and AZA+DFMO.

FIG. 10A-10B. M2 macrophages increase with tumor burden in AZA+DFMO treated mice. FIG. 10A: Representative flow cytometry data shown for one mouse treated with combination AZA+DFMO in vivo. At a later time point, there are significantly increased proportions of M2 macrophages high in CD206 and low in MHC II surface expression. FIG. 10B: Paired tumor burden, represented by ascites volume for the same mouse whose cells are shown in FIG. 10A. The mice's tumor burden is increased at the later time point, coinciding with an increase in M2 macrophages. Please note that this data is shown for only one mouse; thus no statistical analyses were performed.

FIG. 11. Immune cell profiles in 2208L with treatments. TP53−/− mouse mammar models include 2225L, T11 and 2208L. TP53 has been shown to be mutated in ˜40% of breast cancers. These are associated with poor clinical outcomes, and more aggressive molecular subtypes including the basal-like subtype of human breast cancer. TP53−/− or TP53+/− GEM mice develop non-mammary tumors earlier or together with other tumors.

FIG. 12. MDSCs and macrophages in 2208L.

FIG. 13. M- and G-MDSCs and Macrophages in 2208L.

FIG. 14. Table showing tumor types, pathology, latency, molecular subtype and marker expression.

FIG. 15. Combination AZA+DFMO treatment in mice bearing 2208L breast tumors leads to increased survival and decreased tumor burden as measured in weeks 3 through 6 post tumor implant surgery. Single agent AZA treatment had no impact on tumor size in these mice, and no benefit in survival Survival curve (median survival in days); n=4. Significance determined using log-rank Mantel-Cox test.

FIG. 16. Tumor size as measured once weekly in mice bearing 2208L breast tumor xenografts (week 6 post tumor implant). Treatment with DFMO as both a single agent and in combination with AZA maintains low tumor volumes.

FIG. 17. Mature or activated dendritic cells in the tumor microenvironment of the 2208L breast cancer model, shown as a percentage of dendritic cells. AZA treatment increases activation of these cells.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

“Agent” refers to all materials that may be used as or in pharmaceutical compositions, or that may be compounds such as small synthetic or naturally derived organic compounds, nucleic acids, polypeptides, antibodies, fragments, isoforms, variants, or other materials that may be used independently for such purposes, all in accordance with the present invention.

“Antagonist” refers to an agent that down-regulates (e.g., suppresses or inhibits) at least one bioactivity of a protein. An antagonist may be a compound which inhibits or decreases the interaction between a protein and another molecule, e.g., a target peptide or enzyme substrate. An antagonist may also be a compound that down-regulates expression of a gene or which reduces the amount of expressed protein present. The term “inhibitor” is synonymous with the term antagonist.

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies.

The terms “patient,” “individual,” or “subject” are used interchangeably herein, and refer to a mammal, particularly, a human. The patient may have mild, intermediate or severe disease. The patient may be treatment naïve, responding to any form of treatment, or refractory. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters; and primates.

A “small molecule” refers to a composition that has a molecular weight of less than 3 about kilodaltons (kDa), less than about 1.5 kilodaltons, or less than about 1 kilodalton. Small molecules may be nucleic acids, peptides, polypeptides, peptidomimetics, carbohydrates, lipids or other organic (carbon-containing) or inorganic molecules. A “small organic molecule” is an organic compound (or organic compound complexed with an inorganic compound (e.g., metal)) that has a molecular weight of less than about 3 kilodaltons, less than about 1.5 kilodaltons, or less than about 1 kDa.

As used herein, the terms “treatment,” “treating,” “treat” and the like, refer to obtaining a desired pharmacologic and/or physiologic effect. The terms are also used in the context of the administration of a “therapeutically effective amount” of an agent, e.g., an ODC inhibitor, a polyamine transport inhibitor, a DNMTi, an HDACi, a checkpoint inhibitor and/or other immunotherapy. The effect may be prophylactic in terms of completely or partially preventing a particular outcome, disease or symptom thereof and/or may be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease. In particular embodiments, the term is used in the context of treating solid tumors in patients.

As used herein, the term “effective,” means adequate to accomplish a desired, expected, or intended result. More particularly, an “effective amount” or a “therapeutically effective amount” is used interchangeably and refers to an amount of, for example, an ODC inhibitor, a polyamine transport inhibitor, a DNMTi, an HDACi, and/or a checkpoint inhibitor necessary to provide the desired “treatment” (defined herein) or therapeutic effect, e.g., an amount that is effective to prevent, alleviate, treat or ameliorate symptoms of a disease or prolong the survival of the subject being treated. As would be appreciated by one of ordinary skill in the art, the exact amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

The term “combination” refers to two or more therapeutic agents to treat a condition or disorder described herein. Such combination of therapeutic agents may be in the form of a single pill, capsule, or intravenous solution. However, the term “combination” also encompasses the situation when the two or more therapeutic agents are in separate pills, capsules, syringes or intravenous solutions. Likewise, the term “combination therapy” refers to the administration of two or more therapeutic agents to treat a therapeutic condition or disorder described herein. Such administration encompasses co-administration of these therapeutic agents in a substantially simultaneous manner, such as in a single capsule having a fixed ratio of active ingredients or in multiple, or in separate containers (e.g., pills, capsules, etc.) for each active ingredient. In addition, such administration also encompasses use of each type of therapeutic agent in a sequential manner, either at approximately the same time or at different times. In either case, the treatment regimen will provide beneficial effects of the drug combination in treating the conditions or disorders described herein.

The terms “co-administration” and “in combination with” include the administration of two or more therapeutic agents simultaneously, concurrently or sequentially within no specific time limits unless otherwise indicated. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., without limitation, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), essentially concomitantly with, or subsequent to (e.g., without limitation, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

The term “tumor” refers to all neoplastic cell growth and proliferation, whether malignant or benign, and all pre-cancerous and cancerous cells and tissues. As used herein, the term “neoplastic” refers to any form of dysregulated or unregulated cell growth, whether malignant or benign, resulting in abnormal tissue growth. Thus, “neoplastic cells” include malignant and benign cells having dysregulated or unregulated cell growth.

The term “cancer” includes, but is not limited to, solid tumors and blood born tumors. The term “cancer” refers to disease of skin tissues, organs, blood, and vessels, including, but not limited to, cancers of the bladder, bone or blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus.

The term “proliferative” disorder or disease refers to unwanted cell proliferation of one or more subset of cells in a multicellular organism resulting in harm (i.e., discomfort or decreased life expectancy) to the multicellular organism. For example, as used herein, proliferative disorder or disease includes neoplastic disorders and other proliferative disorders.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

The term “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Each carrier must be “acceptable” in the sense of being compatible with the other ingredients of the formulation and not injurious to a subject to whom it is administered. Nor should an acceptable carrier alter the specific activity of the subject compounds.

The term “pharmaceutically acceptable” refers to molecular entities and compositions that are physiologically tolerable and do not typically produce an allergic or similar untoward reaction, such as gastric upset, dizziness and the like, when administered to a human.

The term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

The term “prodrug” means a derivative of a compound that can hydrolyze, oxidize, or otherwise react under biological conditions (in vitro or in vivo) to provide the compound. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, New York 1985).

The term “unit dose” when used in reference to a therapeutic composition refers to physically discrete units suitable as unitary dosage for humans, each unit containing a predetermined quantity of active material calculated to produce the desired therapeutic effect in association with the required diluent; i.e., carrier, or vehicle.

The term “unit-dosage form” refers to a physically discrete unit suitable for administration to a human or animal subject, and packaged individually as is known in the art. Each unit-dose contains a predetermined quantity of an active ingredient(s) sufficient to produce the desired therapeutic effect, in association with the required pharmaceutical carriers or excipients. A unit-dosage form may be administered in fractions or multiples thereof. Examples of a unit-dosage form include an ampoule, syringe, and individually packaged tablet and capsule.

The term “multiple-dosage form” is a plurality of identical unit-dosage forms packaged in a single container to be administered in segregated unit-dosage form. Examples of a multiple-dosage form include a vial, bottle of tablets or capsules, or bottle of pints or gallons.

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer or an isotopic variant of a compound described herein.

As used herein, and unless otherwise specified, a compound described herein is intended to encompass all possible stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence tautomerism in the compound, e.g., that contain an aromatic moiety.

As used herein, and unless otherwise specified, the terms “composition,” “formulation,” and “dosage form” are intended to encompass products comprising the specified ingredient(s) (in the specified amounts, if indicated), as well as any product(s) which result, directly or indirectly, from combination of the specified ingredient(s) in the specified amount(s).

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

### II. Polyamine Reduction Therapy

Polyamines are naturally occurring, polycationic, alkyl amines that are absolute requirements for multiple cellular processes and are particularly important for tumor cell growth (23). Polyamines have an important antioxidant role in the cell and their cationic properties help stabilize newly synthesized and negatively charged DNA. Polyamine reduction therapy includes inhibitors of polyamine biosynthesis (polyamine depletion). In certain embodiments, a polyamine biosynthesis inhibitor inhibits ornithine decarboxylase, an essential enzyme that catalyze the rate limiting step of polyamine synthesis. Polyamine reduction therapy also include inhibitors of polyamine transport. In particular embodiments, polyamine blocking therapy combines inhibition of polyamine biosynthesis with the simultaneous blockade of polyamine transport.

A. Ornithine Decarboxylase Inhibitors

In particular embodiments, the ODC inhibitor comprises difluoromethylornithine or an analog thereof. Alpha-difluoromethylornithine (DFMO; eflornithine) is described in, for example, U.S. Patent Nos. 6,730,809; 6,573,290; 6,258,845; and 4,925,835. The polyamine analogues can include, but are not limited to, N1, N11-bis(ethyl)norspermine (BENSpm) and N¹,N¹²-bis(ethyl)-cis-6,7-dehydrospermine tetrahydrochloride (PG-11047). Ornithine decarboxylase inhibitors are known and described in, for example, U.S. Pat. Nos. 5,753,714; 5,132,293; 5,002,879; 4,720,489; and 4,499,072. Examples include, but are not limited to, alpha-difluoromethylornithine, 2-(difluoromethyl)-2,5-diaminopentanoic acid; alpha-ethynyl ornithine; 6-heptyne-2,5-diamine; 2-methyl-6-heptyne diamine; alpha.-difluoromethyl ornithine; the methyl ester of monofluoromethyl dehydroornithine; the R,R-isomer of methyl acetylenic putrescine, 3-aminooxy-1-aminopropane (APA) and its analogs or derivatives such as CGP 52622A and CGP 54169A, 1,25-dihydroxycholecalciferol, and pharmaceutically acceptable salts and prodrugs thereof. It is understood that the present invention utilizes agents that reduce intracellular polamine pools including, but not limited to, ODC inhibitors. Such agents are used in combination with DNA demethylating agents to enhance tumor response. In further embodiments, compounds that reduce polyamine uptake can be used with DFMO.

The present invention also contemplates the use of inhibitors of other steps in the polyamine synthesis pathway. In one embodiment, the second rate-limiting step—S-adenosylmethionine decarboxylase (AdoMetDC)—can be targeted. One example of an inhibitor include methylgloxal bis(gaunylhydrazone (MGBG). Another compound, based on the structure of MGBG, is 4-amindinoidan-1-one-2′-amidinhydrazone (SAM486A/CCGP48664).

Polyamine reduction therapy also includes inhibitors of the higher polyamine synthases including spermidine and spermine synthase. Such inhibitors include, but are not limited to, S-adenosyl-3-thio-1,8-diaminooctane (AdoDATO), which was designed to specifically inhibit spermidine synthase, and 5-adenosyl-1,12-diamino-3-thio-9-azadodecane (AdoDATAD), which was designed to specifically inhibit spermine synthase. Other inhibitors include deoxyhypusine synthase inhibitors, ODC enzyme induction inhibitors and antizyme inducing agents.

Other inhibitors include polyamine analogues. Natural polaymines such as spermine and spermidine can be used as the original polyamine. Derivatives of spermine include N-(2-mercaptoethyl)spermine-5-carboxamide (MESC), the disulfide from thereof, namely 2,2′-dithiobis(N-ethyl-spermine-5-carboxamide) (DESC), and N-[2,2′-dithio(ethyl,1-aminoethyl)]spermine-5-carboxamide (DEASC). Other polyamine transport inhibitors include lipophilic lysine-spermine conjugates including D-Lys(C(16)acyl)-Spm (Burns et al., 44 J. MED. CHEM. 3632-44 (2001).

B. Polyamine Transport Inhibitors

In certain embodiments, the polyamine transport inhibitor comprises AMXT-1501 (N-(5-amino-6-((3-((4-((3-aminopropyl)amino)butyl)amino)propyl)amino)-6-oxohexyl)palmitamide tetrahydrochloride salt) (Aminex Therapeutics, Inc. (Kirkland, Wash.)).

### III. Epigenetic Therapy

Epigenetic therapy includes DNA methyltransferase inhibitors, histone deactylase inhibitors histone methyltransferase inhibitors and histone demethylase inhibitors.

A. DNA Methyltransferase Inhibitors (DNMTi)

DNMTi useful in the methods provided herein include, but are not limited to, 5-azacytidine (azacytidine), 5-azadeoxycytidine (decitabine; DAC), SGI-110 (guadecitabine) zebulariwne and procaine. In one embodiment, the DNA demethylating agent is 5-azacytidine. 5-azacitidine is 4-amino-1-β-D-ribofuranozyl-s-triazin-2(1H)-one, also known as VIDAZA®. Its empirical formula is C8H₁2N₄O₅, the molecular weight is 244. 5-azacitidine is a white to off-white solid that is insoluble in acetone, ethanol and methyl ketone; slightly soluble in ethanol/water (50/50), propylene glycol and polyethylene glycol; sparingly soluble in water, water-saturated octanol, 5% dextrose in water, N-methyl-2-pyrrolidone, normal saline and 5% Tween 80 in water, and soluble in dimethylsulfoxide (DMSO).

In one embodiment, the methods provided herein comprise administration or co-administration of one or more DNMTi. In one embodiment, the DNA demethylating agents are cytidine analogs. A cytidine analog referred to herein is intended to encompass the free base of the cytidine analog, or a salt, solvate, hydrate, cocrystal, complex, prodrug, precursor, metabolite, and/or derivative thereof. In certain embodiments, a cytidine analog referred to herein encompasses the free base of the cytidine analog, or a salt, solvate, hydrate, cocrystal or complex thereof. In certain embodiments, a cytidine analog referred to herein encompasses the free base of the cytidine analog, or a pharmaceutically acceptable salt, solvate, or hydrate thereof.

In certain embodiments, the cytidine analog is 5-azacytidine (5-azacitidine). In certain embodiments, the cytidine analog is 5-aza-2′-deoxycytidine (decitabine). In certain embodiments, the cytidine analog is 5-azacytidine (5-azacitidine) or 5-aza-2′-deoxycytidine (decitabine). In certain embodiments, the cytidine analog is, for example: 1-β-D-arabinofuranosylcytosine (Cytarabine or ara-C); pseudoiso-cytidine (psi ICR); 5-fluoro-2′-deoxycytidine (FCdR); 2′-deoxy-2′,2′-difluorocytidine (Gemcitabine); 5-aza-2′-deoxy-2′,2′-difluorocytidine; 5-aza-2′-deoxy-2′-fluorocytidine; 1-β-D-ribofuranosyl-2(1H)-pyrimidinone (Zebularine); 2′,3′-dideoxy-5-fluoro-3′-thiacytidine (Emtriva); 2′-cyclocytidine (Ancitabine); 1-β-D-arabinofuranosyl-5-azacytosine (Fazarabine or ara-AC); 6-azacytidine (6-aza-CR); 5,6-dihydro-5-azacytidine (dH-aza-C R); N⁴-pentyloxy-carbonyl-5′-deoxy-5-fluorocytidine (Capecitabine); N⁴-octadecyl-cytarabine; or elaidic acid cytarabine. In certain embodiments, the cytidine analogs provided herein include any compound which is structurally related to cytidine or deoxycytidine and functionally mimics and/or antagonizes the action of cytidine or deoxycytidine.

Certain embodiments herein provide salts, cocrystals, solvates (e.g., hydrates), complexes, prodrugs, precursors, metabolites, and/or other derivatives of the cytidine analogs provided herein. For example, particular embodiments provide salts, cocrystals, solvates (e.g., hydrates), complexes, precursors, metabolites, and/or other derivatives of 5-azacytidine. Certain embodiments herein provide salts, cocrystals, and/or solvates (e.g., hydrates) of the cytidine analogs provided herein. Certain embodiments herein provide salts and/or solvates (e.g., hydrates) of the cytidine analogs provided herein. Certain embodiments provide cytidine analogs that are not salts, cocrystals, solvates (e.g., hydrates), or complexes of the cytidine analogs provided herein. For example, particular embodiments provide 5-azacytidine in a non-ionized, non-solvated (e.g., anhydrous), non-complexed form. Certain embodiments herein provide a mixture of two or more cytidine analogs provided herein.

In one embodiment, the compound used in the methods provided herein is a free base, or a pharmaceutically acceptable salt or solvate thereof. In one embodiment, the free base or the pharmaceutically acceptable salt or solvate is a solid. In another embodiment, the free base or the pharmaceutically acceptable salt or solvate is a solid in an amorphous form. In yet another embodiment, the free base or the pharmaceutically acceptable salt or solvate is a solid in a crystalline form. For example, particular embodiments provide 5-azacytidine in solid forms, which can be prepared, for example, according to the methods described in U.S. Pat. Nos. 6,943,249, 6,887,855 and 7,078,518, and U.S. Patent Application Publication Nos. 2005/027675 and 2006/247189, each of which is incorporated by reference herein in their entireties. In other embodiments, 5-azacytidine in solid forms can be prepared using other methods known in the art.

In one embodiment, the compound used in the methods provided herein is a pharmaceutically acceptable salt of the cytidine analog, which includes, but is not limited to, acetate, adipate, alginate, aspartate, benzoate, benzenesulfonate (besylate), bisulfate, butyrate, citrate, camphorate, camphorsulfonate, cyclopentanepropionate, digluconate, dodecylsulfate, 1,2-ethanedisulfonate (edisylate), ethanesulfonate (esylate), formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate (mesylate), 2-naphthalenesulfonate (napsylate), nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate, or undecanoate salts.

Cytidine analogs provided herein may be prepared using synthetic methods and procedures referenced herein or otherwise available in the literature. For example, particular methods for synthesizing 5-azacytidine are disclosed, e.g., in U.S. Pat. No. 7,038,038 and references discussed therein, each of which is incorporated herein by reference. Other cytidine analogs provided herein may be prepared, e.g., using procedures known in the art, or may be purchased from a commercial source. In one embodiment, the cytidine analogs provided herein may be prepared in a particular solid form (e.g., amorphous or crystalline form). See, e.g., U.S. patent application Ser. No. 10/390,578, filed Mar. 17, 2003 and U.S. patent application Ser. No. 10/390,530, filed Mar. 17, 2003, both of which are incorporated herein by reference in their entireties. In other embodiments, methods of synthesis include methods as disclosed in U.S. Pat. Nos. 7,038,038; 6,887,855; 7,078,518; 6,943,249; and U.S. Ser. No. 10/823,394, all incorporated by reference herein in their entireties.

B. Histone Deacetylase Inhibitors (HDACi)

Histone deacetylases (HDAC) are enzymes capable of removing the acetyl group bound to the lysine residues in the N-terminal portion of histones or in other proteins. HDACs can be subdivided into four classes, on the basis of structural homologies. Class I HDACs (HDAC 1, 2, 3 and 8) are similar to the RPD3 yeast protein and are located in the cell nucleus. Class II HDACs (HDAC 4, 5, 6, 7, 9 and 10) are similar to the HDA1 yeast protein and arc located both in the nucleus and in the cytoplasm. Class III HDACs are a structurally distinct form of NAD-dependent enzymes correlated with the SIR2 yeast protein. Class IV (HDAC 11) consists at the moment of a single enzyme having particular structural characteristics. The HDACs of classes I, II and IV are zinc enzymes and can be inhibited by various classes of molecule: hydroxamic acid derivatives, cyclic tetrapeptides, short-chain fatty acids, aminobenzamides, derivatives of electrophilic ketones, and the like. Class III HDACs are not inhibited by hydroxamic acids, and their inhibitors have structural characteristics different from those of the other classes.

The expression “histone deacetylase inhibitor” in relation to the present invention is to be understood as meaning any molecule of natural, recombinant or synthetic origin capable of inhibiting the activity of at least one of the enzymes classified as HDAC. In particular embodiments, an HDAC inhibitor inhibits enzymes of Class I and II.

Examples of HDACi useful in the compositions and methods of the present invention include, but are not limited to, givinostat, entinostat, trichostatin A (TSA), Vorinostat (SAHA), Valproic Acid (VPA), romidepsin and MS-275. In a specific embodiment, the HDAC inhibitor is givinostat (ITF2357; diethyl-[6-(4-hydroxycarbamoyl-phenylcarbamoyloxymethyl)-naphthalen-2-yl methyl]-ammonium chloride). See, e.g., WO97/43251 (anhydrous form) and in WO2004/065355 (monohydrate crystal form).

HDACi also include chidamide, panohinostat (Farydak, LBH589), belinostat (PXD101), mocetinostat (MGCD0103), abexinostat (PCI-24781), SB939, resminostat (4SC-201), quisinostat (JNJ26481585), Kevetrin, CUDC-101, AR-42, CHR-2845, CHR-3996, 4SC-202, ACY-1215, and ME-344.

Other examples of HDACi include those described in the following patent applications: WO2004/092115, WO2005/019174, WO2003/076422, WO1997/043251, WO2006/010750, WO2006/003068, WO2002/030879, WO2002/022577, WO1993/007148, WO2008/033747, WO2004/069823, EP0847992 and WO2004/071400, the contents of which are incorporated herein by reference in their entirety.

C. Histone Methyltransferase Inhibitors

Histone methyltransferase inhibitors include, but are not limited to, 3-deazaneplanocin A, 3-deazaneplanocin A hydrochloride (DZNep), UNC1999, Chaetocin, Sinefungin, GSK-J1, GSK-J2, GSK-J4, GSK-J5, daminozide, IOX1, Methylstat and JIB-04, BIX-01294, TM2-115, EPZ004777 (Epizyme), EPZ-5676 (pinometostat), 3-Deazaneplanocin (DZNep), GSK343, GSK2816126 (GSK126), EI1 (Novartis), EPZ005687, EPZ-6438, tazemetostat, CPI-1205 (Constellation Pharmaceuticals), CPI-169, tranylcypromine (TCP), ORY-1001 (Oryzon), GSK2879552, 4SC-202, EPT-103182 (Epi Therapeutics), 4-carboxy-2-heterocyclic pyridine derivatives and amino-4-carboxy pyridine derivatives (Quanticel Pharmaceuticals) (see WO2014151106), and derivatives of the foregoing.

D. Histone Demethylase Inhibitors

Histone demethylase inhibitors include, but are not limited to, Ciclopirox, daminozide, GSK-J1, GSK-J2, GSK-J4, GSK LSD1 dihydrochloride, ®-2-Hydroxglutaric acid disodium salt, IOX1, JIB-04, NSC636819, OG-L002, PBIT, RN 1 dihydrochloride, S 2101, TC-E 5002, tranylcypromine hydrochloride, phenelzine, pargyline, WO2012135113A2 (Compounds 29-41), bizine, and derivatives of the foregoing.

### IV. Checkpoint Inhibitors

In another aspect, the present invention provides compositions and methods for treating solid tumors with a combination therapy including a checkpoint inhibitor.

In particular embodiments, the checkpoint inhibitor is a biologic therapeutic or a small molecule. In certain embodiments, the checkpoint inhibitor is a monoclonal antibody, a humanized antibody, a fully human antibody, a fusion protein or a combination thereof. In a particular aspect, the checkpoint inhibitor inhibits a checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In certain embodiments, therapeutic agent is an immunostimulatory agent, a T cell growth factor, an interleukin, an antibody, a vaccine or a combination thereof. In a further aspect, the interleukin is IL-7 or IL-15. In a specific embodiment, the interleukin is glycosylated IL-7. In another embodiment, the vaccine is a dendritic cell vaccine.

In other embodiments, the checkpoint inhibitor is of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof.

Checkpoint inhibitors include any agent that blocks or inhibits the immune system or immune responses. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γΔ, and memory CD8⁺ (αβ) T cells), CD160 (also referred to as BY55), CGEN-15049, CHK 1 and CHK2 kinases, A2aR and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics or small molecules, that bind to and block or inhibit the activity of one or more of CTLA-4, PDL1, PDL2, PD1, BTLA, HVEM, TIM3, GAL9, LAG3, VISTA, KIR, 2B4, CD160 and CGEN-15049. Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor). Checkpoint protein ligands include, but are not limited to PD-L1, PD-L2, B7-H3, B7-H4, CD28, CD86 and TIM-3.

In one specific embodiment, the present invention covers the use of a specific class of checkpoint inhibitors that are drugs that block the interaction between immune checkpoint receptor programmed cell death protein 1 (PD-1) and its ligand PDL-1. See A. Mullard, “New checkpoint inhibitors ride the immunotherapy tsunami,” Nature Reviews: Drug Discovery (2013), 12:489-492. PD-1 is expressed on and regulates the activity of T-cells. Specifically, when PD-1 is unbound to PDL-1, the T-cells can engage and kill target cells. However, when PD-1 is bound to PDL-1 it causes the T-cells to cease engaging and killing target cells. Furthermore, unlike other checkpoints, PD-1 acts proximately. The PDLs are overexpressed directly on cancer cells which leads to increased binding to the PD-1 expressing T-cells.

As used herein, the term “PD-1 antibodies” refers to antibodies that antagonize the activity and/or proliferation of lymphocytes by agonizing PD-1. The term “antagonize the activity” relates to a decrease (or reduction) in lymphocyte proliferation or activity that is at least 5%, 10%, 20%, 30%, 40%, 50%, 60%, 70%, 80%, 90%, or more. The term “antagonize” may be used interchangeably with the terms “inhibitory” and “inhibit”. PD-1-mediated activity can be determined quantitatively using T cell proliferation assays as described herein. There are several PD-1 inhibitors currently being tested in clinical trials including CT-011, BMS 936558, BMS 936559, MK 3475, MPDL 3280A, AMP224, Medi 4736.

One aspect of the present disclosure provides checkpoint inhibitors which are antibodies that can act as agonists of PD-1, thereby modulating immune responses regulated by PD-1. In one embodiment, the anti-PD-1 antibodies can be antigen-binding fragments. Anti-PD-1 antibodies disclosed herein are able to bind to human PD-1 and agonize the activity of PD-1, thereby inhibiting the function of immune cells expressing PD-1.

In one specific embodiment, the present invention covers the use of a specific class of checkpoint inhibitor are drugs that inhibit CTLA-4. Suitable anti-CTLA4 antagonist agents for use in the methods of the invention, include, without limitation, anti-CTLA4 antibodies, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, chimeric anti-CTLA4 antibodies, MDX-010 (ipilimumab), tremelimumab, anti-CD28 antibodies, anti-CTLA4 adnectins, anti-CTLA4 domain antibodies, single chain anti-CTLA4 fragments, heavy chain anti-CTLA4 fragments, light chain anti-CTLA4 fragments, inhibitors of CTLA4 that agonize the co-stimulatory pathway, the antibodies disclosed in PCT Publication No. WO2001/014424, the antibodies disclosed in PCT Publication No. WO2004/035607, the antibodies disclosed in U.S. Publication No. 2005/0201994, and the antibodies disclosed in granted European Patent No. EP1212422 B1. Additional CTLA-4 antibodies are described in U.S. Pat. Nos. 5,811,097, 5,855,887, 6,051,227, and 6,984,720; in PCT Publication Nos. WO01/14424 and WO00/37504; and in U.S. Publication Nos. 2002/0039581 and 2002/086014. Other anti-CTLA-4 antibodies that can be used in a method of the present invention include, for example, those disclosed in: WO98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncology, 22(145):Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281.

Additional anti-CTLA4 antagonists include, but are not limited to, the following: any inhibitor that is capable of disrupting the ability of CD28 antigen to bind to its cognate ligand, to inhibit the ability of CTLA4 to bind to its cognate ligand, to augment T cell responses via the co-stimulatory pathway, to disrupt the ability of B7 to bind to CD28 and/or CTLA4, to disrupt the ability of B7 to activate the co-stimulatory pathway, to disrupt the ability of CD80 to bind to CD28 and/or CTLA4, to disrupt the ability of CD80 to activate the co-stimulatory pathway, to disrupt the ability of CD86 to bind to CD28 and/or CTLA4, to disrupt the ability of CD86 to activate the co-stimulatory pathway, and to disrupt the co-stimulatory pathway, in general from being activated. This necessarily includes small molecule inhibitors of CD28, CD80, CD86, CTLA4, among other members of the co-stimulatory pathway; antibodies directed to CD28, CD80, CD86, CTLA4, among other members of the co-stimulatory pathway; antisense molecules directed against CD28, CD80, CD86, CTLA4, among other members of the co-stimulatory pathway; adnectins directed against CD28, CD80, CD86, CTLA4, among other members of the co-stimulatory pathway, RNAi inhibitors (both single and double stranded) of CD28, CD80, CD86, CTLA4, among other members of the co-stimulatory pathway, among other anti-CTLA4 antagonists.

In one specific embodiment, the present invention covers the use of a specific class of checkpoint inhibitor are drugs that inhibit TIM-3. Blocking the activation of TIM-3 by a ligand, results in an increase in Thl cell activation. Furthermore, TIM-3 has been identified as an important inhibitory receptor expressed by exhausted CD8+ T cells. TIM-3 has also been reported as a key regulator of nucleic acid mediated antitumor immunity. In one example, TIM-3 has been shown to be upregulated on tumor-associated dendritic cells (TADCs).

### V. Pharmaceutical Compositions and Formulations

The pharmaceutical compositions of the present invention are in biologically compatible form suitable for administration in vivo for subjects. The pharmaceutical compositions can further comprise a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which an ODC inhibitor, a polyamine transport inhibitor, a DNMTi, an HDACi, and/or a checkpoint inhibitor are administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like.

The pharmaceutical composition may also contain minor amounts of wetting or emulsifying agents, or pH buffering agents.

The pharmaceutical compositions of the present invention can take the form of solutions, suspensions, emulsions, tablets, pills, capsules, powders, sustained-release formulations and the like. The composition can be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. In a specific embodiment, a pharmaceutical composition comprises an effective amount of an ODC inhibitor and a demethylating agent and optionally a checkpoint inhibitor together with a suitable amount of a pharmaceutically acceptable carrier so as to provide the form for proper administration to the patient. The formulation should suit the mode of administration.

The pharmaceutical compositions of the present invention may be administered by any particular route of administration including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means. Most suitable routes are oral administration or injection.

Optimal precision in achieving concentrations of the therapeutic regimen (e.g., pharmaceutical compositions comprising an ODC inhibitor, a polyamine transport inhibitor, a DNMTi, an HDACi, and/or a checkpoint inhibitor) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical compositions and various therapeutic agents may be independently optimized and combined to achieve a synergistic result wherein the pathology is reduced more than it would be if either was used alone.

In particular, toxicity and therapeutic efficacy of a pharmaceutical composition disclosed herein may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effect is the therapeutic index and it may be expressed as the ratio LD_{50/}ED₅₀. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED₅₀ with little or no toxicity. The dosage may vary within this range depending upon the dosage form employed and the route of administration utilized. For any composition used in the methods of the invention, the therapeutically effective dose may be estimated initially from cell culture assays. A dose may be formulated in animal models to achieve a circulating plasma concentration range that includes the IC₅₀ (the concentration of the test composition that achieves a half-maximal inhibition of symptoms) as determined in cell culture. Such information may be used to accurately determine useful doses in humans. Levels in plasma may be measured, for example, by high performance liquid chromatography.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See WO00/67776, which is entirely expressly incorporated herein by reference.

### VI. Exemplary Methods of Use

In one embodiment, an effective amount of a combination therapy to be used is a therapeutically effective amount. In one embodiment, the amounts of the drugs to be used in the methods provided herein include an amount sufficient to cause improvement in at least a subset of patients with respect to symptoms, overall course of disease, or other parameters known in the art. Precise amounts for therapeutically effective amounts in the pharmaceutical compositions and methods will vary depending on the age, weight, disease, and condition of the patient, as well as the particular drug being administered.

Any suitable daily dose of an ODC inhibitor is contemplated for use with the compositions, dosage forms, and methods disclosed herein. Daily dose of the ODC inhibitor depends on multiple factors, the determination of which is within the skills of one of skill in the art. For example, in certain embodiments, a DFMO dose is 100 milligrams (mg) per kilogram (kg) (45 mg per pound) of body weight.

In particular embodiments, the demethylating agent is administered by, e.g., intravenous (IV), subcutaneous (SC) or oral routes. Certain embodiments herein provide co-administration of the demethylating agent with one or more additional active agents to provide a synergistic therapeutic effect in subjects in need thereof. The co-administered agent(s) may be a cancer therapeutic agent, as described herein. In certain embodiments, the co-administered agent(s) may be dosed, e.g., orally or by injection (e.g., IV or SC).

Certain embodiments herein provide methods for treating solid tumors comprising administering the demethylating agent using, e.g., IV, SC and/or oral administration methods. In certain embodiments, treatment cycles comprise multiple doses administered to a subject in need thereof over multiple days (e.g., 1, 2, 3, 4, 5, 6, 7, 8, 9, 10, 11, 12, 13, 14, or greater than 14 days), optionally followed by treatment dosing holidays (e.g., 1, 2, 3, 4, 5, 6, 7, 8, 9, 10, 11, 12, 13, 14, 15, 16, 17, 18, 19, 20, 21, 22, 23, 24, 25, 26, 27, 28, or greater than 28 days). Suitable dosage amounts for the methods provided herein include, e.g., therapeutically effective amounts and prophylactically effective amounts. For example, in certain embodiments, the amount of the demethylating agent administered in the methods provided herein may range, e.g., between about 30 mg/m²/day and about 2,000 mg/m²/day, between about 100 mg/m²/day and about 1,000 mg/m²/day, between about 100 mg/m²/day and about 500 mg/m²/day, between about 30 mg/m²/day and about 500 mg/m²/day, between about 30 mg/m²/day and about 200 mg/m²/day, between about 30 mg/m²/day and about 100 mg/m²/day, between about 30 mg/m²/day and about 75 mg/m²/day, or between about 120 mg/m²/day and about 250 mg/m²/day. In certain embodiments, particular dosages are, e.g., about 30 mg/m²/day, about 40 mg/m²/day, about 50 mg/m²/day, about 60 mg/m²/day, about 75 mg/m²/day, about 80 mg/m²/day, about 100 mg/m²/day, about 120 mg/m²/day, about 140 mg/m²/day, about 150 mg/m²/day, about 180 mg/m²/day, about 200 mg/m²/day, about 220 mg/m²/day, about 240 mg/m²/day, about 250 mg/m²/day, about 260 mg/m²/day, about 280 mg/m²/day, about 300 mg/m²/day, about 320 mg/m²/day, about 350 mg/m²/day, about 380 mg/m²/day, about 400 mg/m²/day, about 450 mg/m²/day, or about 500 mg/m²/day. In certain embodiments, particular dosages are, e.g., up to about 30 mg/m²/day, up to about 40 mg/m²/day, up to about 50 mg/m²/day, up to about 60 mg/m²/day, up to about 70 mg/m²/day, up to about 80 mg/m²/day, up to about 90 mg/m²/day, up to about 100 mg/m²/day, up to about 120 mg/m²/day, up to about 140 mg/m²/day, up to about 150 mg/m²/day, up to about 180 mg/m²/day, up to about 200 mg/m²/day, up to about 220 mg/m²/day, up to about 240 mg/m²/day, up to about 250 mg/m²/day, up to about 260 mg/m²/day, up to about 280 mg/m²/day, up to about 300 mg/m²/day, up to about 320 mg/m²/day, up to about 350 mg/m²/day, up to about 380 mg/m²/day, up to about 400 mg/m²/day, up to about 450 mg/m²/day, up to about 500 mg/m²/day, up to about 750 mg/m²/day, or up to about 1000 mg/m²/day. In a specific non-limiting embodiment, the dose of the demethylating agent is about 40 mg/m².

In one embodiment, the amount of the demethylating agent administered in the methods provided herein may range, e.g., between about 5 mg/day and about 2,000 mg/day, between about 10 mg/day and about 2,000 mg/day, between about 20 mg/day and about 2,000 mg/day, between about 50 mg/day and about 1,000 mg/day, between about 100 mg/day and about 1,000 mg/day, between about 100 mg/day and about 500 mg/day, between about 150 mg/day and about 500 mg/day, or between about 150 mg/day and about 250 mg/day. In certain embodiments, particular dosages are, e.g., about 10 mg/day, about 20 mg/day, about 50 mg/day, about 75 mg/day, about 100 mg/day, about 120 mg/day, about 150 mg/day, about 200 mg/day, about 250 mg/day, about 300 mg/day, about 350 mg/day, about 400 mg/day, about 450 mg/day, about 500 mg/day, about 600 mg/day, about 700 mg/day, about 800 mg/day, about 900 mg/day, about 1,000 mg/day, about 1,200 mg/day, or about 1,500 mg/day. In certain embodiments, particular dosages are, e.g., up to about 10 mg/day, up to about 20 mg/day, up to about 50 mg/day, up to about 75 mg/day, up to about 100 mg/day, up to about 120 mg/day, up to about 150 mg/day, up to about 200 mg/day, up to about 250 mg/day, up to about 300 mg/day, up to about 350 mg/day, up to about 400 mg/day, up to about 450 mg/day, up to about 500 mg/day, up to about 600 mg/day, up to about 700 mg/day, up to about 800 mg/day, up to about 900 mg/day, up to about 1,000 mg/day, up to about 1,200 mg/day, or up to about 1,500 mg/day.

In one embodiment, the amount of the demethylating agent in the pharmaceutical composition or dosage form provided herein may range, e.g., between about 5 mg and about 2,000 mg, between about 10 mg and about 2,000 mg, between about 20 mg and about 2,000 mg, between about 30 mg and about 1,000 mg, between about 30 mg and about 500 mg, between about 30 mg and about 250 mg, between about 100 mg and about 500 mg, between about 150 mg and about 500 mg, or between about 150 mg and about 250 mg. In certain embodiments, particular amounts are, e.g., about 10 mg, about 20 mg, about 30 mg, about 40 mg, about 50 mg, about 75 mg, about 100 mg, about 120 mg, about 150 mg, about 200 mg, about 250 mg, about 300 mg, about 350 mg, about 400 mg, about 450 mg, about 500 mg, about 600 mg, about 700 mg, about 800 mg, about 900 mg, about 1,000 mg, about 1,200 mg, or about 1,500 mg. In certain embodiments, particular amounts are, e.g., up to about 10 mg, up to about 20 mg, up to about 30 mg, up to about 40 mg, up to about 50 mg, up to about 75 mg, up to about 100 mg, up to about 120 mg, up to about 150 mg, up to about 200 mg, up to about 250 mg, up to about 300 mg, up to about 350 mg, up to about 400 mg, up to about 450 mg, up to about 500 mg, up to about 600 mg, up to about 700 mg, up to about 800 mg, up to about 900 mg, up to about 1,000 mg, up to about 1,200 mg, or up to about 1,500 mg.

In one embodiment, depending on the disease to be treated and the subject's condition, the demethylating agent may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. The demethylating agent may be formulated, alone or together with one or more active agent(s), in suitable dosage unit with pharmaceutically acceptable excipients, carriers, adjuvants and vehicles, appropriate for each route of administration. In one embodiment, the demethylating agent is administered orally. In another embodiment, the demethylating agent is administered parenterally. In yet another embodiment, the demethylating agent is administered intravenously.

In one embodiment, the demethylating agent can be delivered as a single dose such as, e.g., a single bolus injection, or oral tablets or pills; or over time such as, e.g., continuous infusion over time or divided bolus doses over time. In one embodiment, the demethylating agent can be administered repetitively if necessary, for example, until the patient experiences stable disease or regression, or until the patient experiences disease progression or unacceptable toxicity. For example, stable disease for solid tumors generally means that the perpendicular diameter of measurable lesions has not increased by 25% or more from the last measurement. See, e.g., Response Evaluation Criteria in Solid Tumors (RECIST) Guidelines, Journal of the National Cancer Institute 92(3): 205-216 (2000). Stable disease or lack thereof is determined by methods known in the art such as evaluation of patient's symptoms, physical examination, visualization of the tumor that has been imaged using X-ray, CAT, PET, or MRI scan and other commonly accepted evaluation modalities.

In one embodiment, the demethylating agent can be administered once daily or divided into multiple daily doses such as twice daily, three times daily, and four times daily. In one embodiment, the administration can be continuous (i.e., daily for consecutive days or every day), intermittent, e.g., in cycles (i.e., including days, weeks, or months of rest when no drug is administered). In one embodiment, the demethylating agent is administered daily, for example, once or more than once each day for a period of time. In one embodiment, the demethylating agent is administered daily for an uninterrupted period of at least 7 days, in some embodiments, up to 52 weeks. In one embodiment, the demethylating agent is administered intermittently, i.e., stopping and starting at either regular or irregular intervals. In one embodiment, the demethylating agent is administered for one to six days per week. In one embodiment, the demethylating agent is administered in cycles (e.g., daily administration for two to eight consecutive weeks, then a rest period with no administration for up to one week; or e.g., daily administration for one week, then a rest period with no administration for up to three weeks). In one embodiment, the demethylating agent is administered on alternate days. In one embodiment, the demethylating agent is administered in cycles (e.g., administered daily or continuously for a certain period interrupted with a rest period).

In one embodiment, the frequency of administration ranges from about daily to about monthly. In certain embodiments, the demethylating agent is administered once a day, twice a day, three times a day, four times a day, once every other day, twice a week, once every week, once every two weeks, once every three weeks, or once every four weeks. In one embodiment, the demethylating agent is administered once a day. In another embodiment, the demethylating agent is administered twice a day. In yet another embodiment, the demethylating agent is administered three times a day. In still another embodiment, the demethylating agent is administered four times a day.

In one embodiment, the demethylating agent is administered once per day from one day to six months, from one week to three months, from one week to four weeks, from one week to three weeks, or from one week to two weeks. In certain embodiments, the demethylating agent is administered once per day for one week, two weeks, three weeks, or four weeks. In one embodiment, the demethylating agent is administered once per day for one week. In another embodiment, the demethylating agent is administered once per day for two weeks. In yet another embodiment, the demethylating agent is administered once per day for three weeks. In still another embodiment, the demethylating agent is administered once per day for four weeks.

In one embodiment, the demethylating agent is administered once per day for about 1 week, about 2 weeks, about 3 weeks, about 4 weeks, about 6 weeks, about 9 weeks, about 12 weeks, about 15 weeks, about 18 weeks, about 21 weeks, or about 26 weeks. In certain embodiments, the demethylating agent is administered intermittently. In certain embodiments, the demethylating agent is administered intermittently in the amount of between about 30 mg/m²/day and about 2,000 mg/m²/day. In certain embodiments, the demethylating agent is administered continuously. In certain embodiments, the demethylating agent is administered continuously in the amount of between about 30 mg/m²/day and about 1,000 mg/m²/day.

In certain embodiments, the demethylating agent is administered to a patient in cycles (e.g., daily administration for one week, then a rest period with no administration for up to three weeks). Cycling therapy involves the administration of an active agent for a period of time, followed by a rest for a period of time, and repeating this sequential administration. Cycling therapy can reduce the development of resistance, avoid or reduce the side effects, and/or improves the efficacy of the treatment.

In one embodiment, the demethylating agent is administered to a patient in cycles. In one embodiment, a method provided herein comprises administering the demethylating agent in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or greater than 40 cycles. In one embodiment, the median number of cycles administered in a group of patients is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or greater than about 30 cycles.

In one embodiment, the demethylating agent is administered to a patient at a dose provided herein over a cycle of 28 days which consists of a 7-day treatment period and a 21-day resting period. In one embodiment, the demethylating agent is administered to a patient at a dose provided herein each day from day 1 to day 7, followed with a resting period from day 8 to day 28 with no administration of the demethylating agent. In one embodiment, the demethylating agent is administered to a patient in cycles, each cycle consisting of a 7-day treatment period followed with a 21-day resting period. In particular embodiments, the demethylating agent is administered to a patient at a dose of about 50, about 60, about 70, about 75, about 80, about 90, or about 100 mg/m²/d, for 7 days, followed with a resting period of 21 days. In one embodiment, the demethylating agent is administered intravenously. In one embodiment, the demethylating agent is administered subcutaneously.

In other embodiments, the demethylating agent is administered orally in cycles.

Accordingly, in one embodiment, the demethylating agent is administered daily in single or divided doses for about one week, about two weeks, about three weeks, about four weeks, about five weeks, about six weeks, about eight weeks, about ten weeks, about fifteen weeks, or about twenty weeks, followed by a rest period of about 1 day to about ten weeks. In one embodiment, the methods provided herein contemplate cycling treatments of about one week, about two weeks, about three weeks, about four weeks, about five weeks, about six weeks, about eight weeks, about ten weeks, about fifteen weeks, or about twenty weeks. In some embodiments, the demethylating agent is administered daily in single or divided doses for about one week, about two weeks, about three weeks, about four weeks, about five weeks, or about six weeks with a rest period of about 1, 3, 5, 7, 9, 12, 14, 16, 18, 20, 22, 24, 26, 28, 29, or 30 days. In some embodiments, the rest period is 1 day. In some embodiments, the rest period is 3 days. In some embodiments, the rest period is 7 days. In some embodiments, the rest period is 14 days. In some embodiments, the rest period is 28 days. The frequency, number and length of dosing cycles can be increased or decreased.

In one embodiment, the methods provided herein comprise: i) administering to the subject a first daily dose of the demethylating agent; ii) optionally resting for a period of at least one day where the demethylating agent is not administered to the subject; iii) administering a second dose of the demethylating agent to the subject; and iv) repeating steps ii) to iii) a plurality of times. In certain embodiments, the first daily dose is between about 30 mg/m²/day and about 2,000 mg/m²/day. In certain embodiments, the second daily dose is between about 30 mg/m²/day and about 2,000 mg/m²/day. In certain embodiments, the first daily dose is higher than the second daily dose. In certain embodiments, the second daily dose is higher than the first daily dose. In one embodiment, the rest period is 2 days, 3 days, 5 days, 7 days, 10 days, 12 days, 13 days, 14 days, 15 days, 17 days, 21 days, or 28 days. In one embodiment, the rest period is at least 2 days and steps ii) through iii) are repeated at least three times. In one embodiment, the rest period is at least 2 days and steps ii) through iii) are repeated at least five times. In one embodiment, the rest period is at least 3 days and steps ii) through iii) are repeated at least three times. In one embodiment, the rest period is at least 3 days and steps ii) through iii) are repeated at least five times. In one embodiment, the rest period is at least 7 days and steps ii) through iii) are repeated at least three times. In one embodiment, the rest period is at least 7 days and steps ii) through iii) are repeated at least five times. In one embodiment, the rest period is at least 14 days and steps ii) through iii) are repeated at least three times. In one embodiment, the rest period is at least 14 days and steps ii) through iii) are repeated at least five times. In one embodiment, the rest period is at least 21 days and steps ii) through iii) are repeated at least three times. In one embodiment, the rest period is at least 21 days and steps ii) through iii) are repeated at least five times. In one embodiment, the rest period is at least 28 days and steps ii) through iii) are repeated at least three times. In one embodiment, the rest period is at least 28 days and steps ii) through iii) are repeated at least five times. In one embodiment, the methods provided herein comprise: i) administering to the subject a first daily dose of the demethylating agent for 1, 2, 3, 4, 5, 6, 7, 8, 9, 10, 11, 12, 13, or 14 days; ii) resting fora period of 1, 2, 3, 4, 5, 6, 7, 8, 9, 10, 11, 12, 13, 14, 15, 16, 17, 18, 19, 20, 21, 22, 23, 24, 25, 26, 27, or 28 days; iii) administering to the subject a second daily dose of the demethylating agent for 1, 2, 3, 4, 5, 6, 7, 8, 9, 10, 11, 12, 13, or 14 days; and iv) repeating steps ii) to iii) a plurality of times. In one embodiment, the methods provided herein comprise: i) administering to the subject a daily dose of the demethylating agent for 1, 2, 3, 4, 5, 6, 7, 8, 9, 10, 11, 12, 13, or 14 days; ii) resting for a period of 1, 2, 3, 4, 5, 6, 7, 8, 9, 10, 11, 12, 13, 14, 15, 16, 17, 18, 19, 20, 21, 22, 23, 24, 25, 26, 27, or 28 days; and iii) repeating steps i) to ii) a plurality of times. In one embodiment, the methods provided herein comprise: i) administering to the subject a daily dose of the demethylating agent for 7 days; ii) resting for a period of 21 days; and iii) repeating steps i) to ii) a plurality of times. In one embodiment, the daily dose is between about 30 mg/m²/day and about 2,000 mg/m²/day. In one embodiment, the daily dose is between about 30 mg/m²/day and about 1,000 mg/m²/day. In one embodiment, the daily dose is between about 30 mg/m²/day and about 500 mg/m²/day. In one embodiment, the daily dose is between about 30 mg/m²/day and about 200 mg/m²/day. In one embodiment, the daily dose is between about 30 mg/m²/day and about 100 mg/m²/day.

In certain embodiments, the demethylating agent is administered continuously for between about 1 and about 52 weeks. In certain embodiments, the demethylating agent is administered continuously for about 1, 2, 3, 4, 5, 6, 7, 8, 9, 10, 11, or 12 months. In certain embodiments, the demethylating agent is administered continuously for about 14, about 28, about 42, about 84, or about 112 days. It is understood that the duration of the treatment may vary with the age, weight, and condition of the subject being treated, and may be determined empirically using known testing protocols or according to the professional judgment of the person providing or supervising the treatment. The skilled clinician will be able to readily determine, without undue experimentation, an effective drug dose and treatment duration, for treating an individual subject having a particular type of cancer.

In one embodiment, pharmaceutical compositions may contain sufficient quantities of the demethylating agent to provide a daily dosage of about 10 to 150 mg/m² (based on patient body surface area) or about 0.1 to 4 mg/kg (based on patient body weight) as single or divided (2-3) daily doses. In one embodiment, dosage is provided via a seven-day administration of 75 mg/m² subcutaneously, once every twenty-eight days, for as long as clinically necessary. In one embodiment, dosage is provided via a seven-day administration of 100 mg/m² subcutaneously, once every twenty-eight days, for as long as clinically necessary. In one embodiment, up to 4, up to 5, up to 6, up to 7, up to 8, up to 9 or more 28-day cycles are administered. Other methods for providing an effective amount of the demethylating agent are disclosed in, for example, “Colon-Targeted Oral Formulations of Cytidine Analogs”, U.S. Ser. No. 11/849,958, and “Oral Formulations of Cytidine Analogs and Methods of Use Thereof”, U.S. Ser. No. 12/466,213, both of which are incorporated by reference herein in their entireties.

In particular embodiments, the number of cycles administered is, e.g., at least 4, at least 5, at least 6, at least 7, at least 8, at least 9, at least 10, at least 11, at least 12, at least 13, at least 14, at least 15, at least 16, at least 17, at least 18, at least 19, at least 20, at least 22, at least 24, at least 26, at least 28, at least 30, at least 32, at least 34, at least 36, at least 38, at least 40, at least 42, at least 44, at least 46, at least 48, or at least 50 cycles of the demethylating agent treatment. In particular embodiments, the treatment is administered, e.g., 1, 2, 3, 4, 5, 6, 7, 8, 9, 10, 11, 12, 13, or 14 days out of a 28-day period. In particular embodiments, the demethylating agent dose is, e.g., at least 10 mg/day, at least 20 mg/day, at least 30 mg/day, at least 40 mg/day, at least 50 mg/day, at least 55 mg/day, at least 60 mg/day, at least 65 mg/day, at least 70 mg/day, at least 75 mg/day, at least 80 mg/day, at least 85 mg/day, at least 90 mg/day, at least 95 mg/day, or at least 100 mg/day.

In particular embodiments, the dosing is performed, e.g., subcutaneously or intravenously. In particular embodiments, the contemplated specific the demethylating agent dose is, e.g., at least 30 mg/m²/day, at least 40 mg/m²/day, at least 50 mg/m²/day, at least 60 mg/m²/day, at least 70 mg/m²/day, at least 75 mg/m²/day, at least 80 mg/m2/day, at least 90 mg/m²/day, or at least 100 mg/m²/day. One particular embodiment herein provides administering the treatment for 7 days out of each 28-day period. One particular embodiment herein provides a dosing regimen of 75 mg/m² subcutaneously or intravenously, daily for 7 days. One particular embodiment herein provides a dosing regimen of 100 mg/m² subcutaneously or intravenously, daily for 7 days.

In one embodiment, an HDAC inhibitor (e.g., givinostat, entinostat, romidepsin and the like) is administered intravenously. In one embodiment, the HDAC inhibitor is administered intravenously over a 1-6 hour period. In one embodiment, the HDAC inhibitor is administered intravenously over a 3-4 hour period. In one embodiment, the HDAC inhibitor is administered intravenously over a 5-6 hour period. In one embodiment, the HDAC inhibitor is administered intravenously over a 4 hour period.

In one embodiment, the HDAC inhibitor is administered in a dose ranging from 0.5 mg/m² to 28 mg/m². In one embodiment, the HDAC inhibitor is administered in a dose ranging from 0.5 mg/m² to 5 mg/m². In one embodiment, the HDAC inhibitor is administered in a dose ranging from 1 mg/m² to 25 mg/m². In one embodiment, the HDAC inhibitor is administered in a dose ranging from 1 mg/m² to 20 mg/m². In one embodiment, the HDAC inhibitor is administered in a dose ranging from 1 mg/m² to 15 mg/m². In one embodiment, the HDAC inhibitor is administered in a dose ranging from 2 mg/m² to 15 mg/m². In one embodiment, the HDAC inhibitor is administered in a dose ranging from 2 mg/m² to 12 mg/m². In one embodiment, the HDAC inhibitor is administered in a dose ranging from 4 mg/m² to 12 mg/m². In one embodiment, the HDAC inhibitor is administered in a dose ranging from 6 mg/m² to 12 mg/m². In one embodiment, the HDAC inhibitor is administered in a dose ranging from 8 mg/m² to 12 mg/m². In one embodiment, the HDAC inhibitor is administered in a dose ranging from 8 mg/m² to 10 mg/m². In one embodiment, the HDAC inhibitor is administered in a dose of about 8 mg/m². In one embodiment, the HDAC inhibitor is administered in a dose of about 9 mg/m². In one embodiment, the HDAC inhibitor is administered in a dose of about 10 mg/m². In one embodiment, the HDAC inhibitor is administered in a dose of about 11 mg/m². In one embodiment, the HDAC inhibitor is administered in a dose of about 12 mg/m². In one embodiment, the HDAC inhibitor is administered in a dose of about 13 mg/m². In one embodiment, the HDAC inhibitor is administered in a dose of about 14 mg/m². In one embodiment, the HDAC inhibitor is administered in a dose of about 15 mg/m².

In one embodiment, the HDAC inhibitor is administered in a dose of 14 mg/m² over a 4 hour iv infusion on days 1, 8 and 15 of the 28 day cycle. In one embodiment, the cycle is repeated every 28 days.

In one embodiment, increasing doses of the HDAC inhibitor are administered over the course of a cycle. In one embodiment, the dose of about 8 mg/m² followed by a dose of about 10 mg/m², followed by a dose of about 12 mg/m² is administered over a cycle.

In one embodiment, the HDAC inhibitor is administered orally. In one embodiment, the HDAC inhibitor is administered in a dose ranging from 10 mg/m² to 300 mg/m². In one embodiment, the HDAC inhibitor is administered in a dose ranging from 15 mg/m² to 250 mg/m². In one embodiment, the HDAC inhibitor is administered in a dose ranging from 20 mg/m² to 200 mg/m². In one embodiment, the HDAC inhibitor is administered in a dose ranging from 25 mg/m² to 150 mg/m². In one embodiment, the HDAC inhibitor is administered in a dose ranging from 25 mg/m² to 100 mg/m². In one embodiment, the HDAC inhibitor is administered in a dose ranging from 25 mg/m² to 75 mg/m².

In one embodiment, the HDAC inhibitor is administered orally on a daily basis. In one embodiment, the HDAC inhibitor is administered orally every other day. In one embodiment, the HDAC inhibitor is administered orally every third, fourth, fifth, or sixth day. In one embodiment, the HDAC inhibitor is administered orally every week. In one embodiment, the HDAC inhibitor is administered orally every other week. Merck's ZOLINZA® (vorinostat) is administered 400 mg orally once daily with food.

Any suitable daily dose of a checkpoint inhibitor is contemplated for use with the compositions, dosage forms, and methods disclosed herein. Daily dose of the checkpoint inhibitor depends on multiple factors, the determination of which is within the skills of one of skill in the art. For example, the daily dose of the checkpoint inhibitor depends on the strength of the checkpoint inhibitor. Weak immune checkpoint inhibitors will require higher daily doses than moderate immune checkpoint inhibitors, and moderate immune checkpoint inhibitors will require higher daily doses than strong immune checkpoint inhibitors. For example, Merck's pembrolizumab (Keytruda) is approved for 2 mg/kg iv over 30 minutes every three weeks (50 mg lyophilized power). Nivolumab (OPDVO) is administered 3 mg/kg iv over 60 minutes every 2 weeks (injection dosage form: 40 mg/4 ml and 100 mg/10/ml in single use vial). Ipilimumab (YERVOY) is administered 3 mg/kg iv over 90 minutes every 3 weeks for a total of 4 doses (dosage form: 50 mg/10 ml, 200 mg/40 ml).

### VII. Kits

In other embodiments, kits are provided. Kits according to the invention include package(s) comprising compounds or compositions of the invention. In a specific embodiment, a kit comprises a polyamine reduction therapy and an epigenetic therapy. In a more specific embodiment, the polyamine reduction therapy comprises a polyamine synthesis inhibitor or a polyamine transport inhibitor. In another specific embodiment, the epigenetic therapy comprises a DNMTi or an HDACi. The kit can further comprise a checkpoint inhibitor and/or an agent that reduces M2 macrophages.

The phrase “package” means any vessel containing compounds or compositions presented herein. In some embodiments, the package can be a box or wrapping. Packaging materials for use in packaging pharmaceutical products are well-known to those of skill in the art. Examples of pharmaceutical packaging materials include, but are not limited to, bottles, tubes, inhalers, pumps, bags, vials, containers, syringes, bottles, and any packaging material suitable for a selected formulation and intended mode of administration and treatment.

The kit can also contain items that are not contained within the package, but are attached to the outside of the package, for example, pipettes.

Kits can further contain instructions for administering compounds or compositions of the invention to a patient. Kits also can comprise instructions for approved uses of compounds herein by regulatory agencies, such as the United States Food and Drug Administration. Kits can also contain labeling or product inserts for the compounds. The package(s) and/or any product insert(s) may themselves be approved by regulatory agencies. The kits can include compounds in the solid phase or in a liquid phase (such as buffers provided) in a package. The kits can also include buffers for preparing solutions for conducting the methods, and pipettes for transferring liquids from one container to another.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Although ovarian cancer has a low incidence rate, it remains the most deadly gynecologic malignancy. Previous work has demonstrated that the DNMTi 5-Azacytidine (AZA) activates type I interferon signaling to increase IFNγ+ T cells and NK cells and reduce the percentage of macrophages in the tumor microenvironment. To improve the efficacy of epigenetic therapy, we hypothesized that the addition of a-difluoromethylornithine (DFMO), an ornithine decarboxylase inhibitor, may further decrease immunosuppressive cell populations improving outcome. We tested this hypothesis in an immunocompetent mouse model for ovarian cancer and found that in vivo, AZA and DFMO, either alone or in combination, significantly increased survival, decreased tumor burden, and caused recruitment of activated (IFNγ+) CD4+ T cells, CD8+ T cells, and NK cells. The combination therapy had a striking increase in survival when compared to single agent treatment, despite a smaller difference in recruited lymphocytes. Instead, combination therapy led to a significant decrease in immunosuppressive cells such as M2 polarized macrophages and an increase in tumor-killing M1 macrophages. In this model, depletion of macrophages with a CSF1R-blocking antibody reduced the efficacy of AZA+DFMO treatment and resulted in fewer M1 macrophages in the tumor microenvironment. These observations suggest our novel combination therapy modifies macrophage polarization in the tumor microenvironment, recruiting M1 macrophages and prolonging survival.

### Materials and Methods

Drugs and reagents. DFMO was kindly provided by Dr. Patrick Woster (Medical University of South Carolina). AZA was purchased from Sigma-Aldrich (Catalog No. 320-67-2). α-PD-1 was kindly provided by the Michael Lim lab. α-CSF1R (BioXCell Clone AFS98) was generously provided by Janssen.

Animals. Female C57BL/6NHsd wild-type (WT) mice (7-8 wk old) were purchased from Envigo International Holdings, Inc. (Indianapolis, Ind.). Mice were housed at the Johns Hopkins Kimmel Cancer Center Animal Resources Core and cared for in accordance with the policies of The Johns Hopkins University Animal Care and Use Committee and our approved animal protocol.

Syngeneic mouse model. 250,000 VEGF-β-Defensin ID8 (VDID8) syngeneic mouse ovarian surface epithelial (MOSE) cells were injected intraperitoneally into wild-type (WT) C57BL/6 mice. Cells were obtained from Dr. Chien-Fu Hung and tested for Mycoplasma every 6 months using MycoAlert PLUS (Lonza LT07-701) per manufacturer's instructions and as previously described (18). Dr. Katherine Roby developed the ID8 model via mild trypsinization of the ovarian surface epithelium, followed by long-term passage in vitro until the cells spontaneously immortalized (34). The parental ID8 clone has been further modified to enhance its usefulness as a tool by overexpressing VEGF and β-defensin, making the tumor more aggressive and immunosuppressive (35). The VDID8 cells are also positive for Luciferase and green fluorescent protein (GFP). While this model has proven to be an excellent research tool, it has limitations in representing high-grade serous ovarian cancer in humans because it is derived from mouse ovarian surface epithelium, not the fallopian tube, and is Trp53 wildtype. In mice however, ovarian cancer can arise from either fallopian tube epithelium (FTE) or ovarian surface epithelium (OSE) and ID8 is the most widely used MOSE model for immunotherapy studies in ovarian cancer.

Mice were treated with 0.5 mg/kg AZA/saline, Monday through Friday, every other week and continuous 2% DFMO in drinking water. 200 ug of α-PD-1 or IgG was injected i.p. 4 times total on days 17, 20, 24, and 27 post i.p. injection of VDID8 cells. 200 ug of α-CSF1R or IgG was injected i.p. twice weekly beginning two weeks prior to VDID8 cell injection, and continuing throughout the duration of the experiment.

Ascites tissue harvest and processing. When ascites fluid is collected from the mice, the cells obtained represent the tumor microenvironment and can be further analyzed to help illustrate the mixed population of cells surrounding the tumor. Ascites was collected, filtered, incubated in ACK buffer (Quality Biological) to lyse red blood cells, and washed. The mononuclear cells collected were then cultured for four hours in RPMI (Corning) with 10% FBS in the presence of phorbol 12-myristate 13-acetate (PMA) and ionomycin to stimulate cells, and brefeldin A and monensin (Invitrogen 00-4975-93) to cause aggregation of secreted proteins inside the cell.

Flow cytometry. Cells were washed and blocked with FcR Blocking Reagent (Miltenyi Biotec 130-092-575) and stained for cell-surface markers including Live/Dead (eBioscience 65-0865-14), CD45 (BD Biosciences 563891), CD3 (BD Biosciences 560527), CD4 (BD Biosciences 563331), CD8 (BD Biosciences 563152), NK1.1 (BD Biosciences 562921), F4/80 (BioLegend 123113), CD11b (BioLegend 101222), MHC II (isotype control 400627; BioLegend 107619), CD206 (BioLegend 141708), CD11c (BD Biosciences 564079), Ly6C (BD Biosciences 562728), Ly6G (BD Biosciences 563005), CD80 (BD Biosciences 553769), and CD86 (BD Biosciences 558703). Cells were permeabilized and stained for intracellular IFNγ (isotype control 554686; BD Biosciences 554413). Flow cytometry acquisition was performed on an LSR II cytometer (BD Biosciences), and data were analyzed using FlowJo software version 10.2.

Flow sorting. Lysed and processed bulk ascites cells were blocked with FcR Blocking Reagent (Miltenyi Biotec 130-092-575) and stained for cell-surface markers including Live/Dead (eBioscience 65-0865-14), CD45 (BD Biosciences 563891), F4/80 (BioLegend 123113), CD11b (BioLegend 101222), MHC II (isotype control 400627; BioLegend 107619), CD206 (BioLegend 141708), and CD11c (BD Biosciences 564079). Prepared cells were suspended in PBS and sorted immediately on a BSL-2 FACSAria II. M1 macrophages were sorted on a gate as follows: CD45+ L/D− F4\80+ CD11b+ MHC II+ CD206− CD11c−. M2 macrophages were sorted on a gate as follows: CD45+ L/D− F4\80+ CD11b+ MHC II− CD206+ CD11c−.

RNA isolation and quantitative reverse-transcriptase PCR. Total RNA was isolated from sorted macrophages using TRIzol reagent according to the manufacturer's protocol (Invitrogen, Carlsbad, Calif.). 200 ng of RNA was used for cDNA synthesis using qScript cDNA SuperMix (Quanta Biosciences, Gaithersburg, Md.), followed by SYBR green-mediated real-time PCR (Universal SYBR Green Supermix, BioRad, Hercules, Calif.) using custom primers specific for Arg1, Fizz1, and iNOS2.

(Arg1 F:
(SEQ ID NO: 1)
CAGAAGAATGGAAGAGTCAG;
Arg1 R:
(SEQ ID NO: 2)
CAGATATGCAGGCAGGGAGTCACC;
Fizz1 F:
(SEQ ID NO: 3)
GGTCCCAGTGCATATGGATGAGACCA;
Fizz1 R:
(SEQ ID NO: 4)
CACCTCTTCACTCGAGGGACAGTTGG;
iNOS2 F:
(SEQ ID NO: 5)
CCGAAGCAAACATCACATTCA;
iNOS2 R:
(SEQ ID NO: 6)
GGTCTAAAGGCTCCGGGCT).

In each experiment, samples were performed in duplicate, normalized to (3-actin as an internal control, and fold change in expression relative to M1 or M2 macrophage was determined using the 2-ΔΔCt algorithm. Thermocycling was performed on a Bio-Rad iQ2 real-time PCR detection system and data collected using the iQ5 optical system software.

ELISA assays. Bulk ascites fluid collected from individual treated mice was centrifuged at low speed (1000 rpm) for 15 minutes, and 1,000 uL of supernatant was collected and stored at −80C. Circulating CSF-1 levels in mice treated with IgG vs. CSF1R was detected using an ELISA kit (R&D Systems Kit #MMC00) according to instructions.

Polyamines. Polyamines were analyzed via high-performance liquid chromatography (HPLC) as previously described (36).

Statistical analysis. Data were graphed in GraphPad Prism 7.0 and tested for a Gaussian distribution using the Shapiro-Wilk test. Significance was determined for sets of data with more than two groups using the one-way ANOVA or Kruskal Wallis test dependent upon normality results from the Shapiro-Wilk test. If only two sets of data were compared, either the Mann-Whitney (non-parametric) or student's t test (parametric) were used dependent on normality results. Significances in survival data were determined by Mantel-Cox (log-rank) test. P values less than 0.05 were deemed significant. Outliers were removed from ascites volume datasets and ascites immune cell datasets using Peirce's criterion (37). Significances are shown as *P<0.05, **P<0.01, and ***P<0.001 ****P<0.0001.

### Results

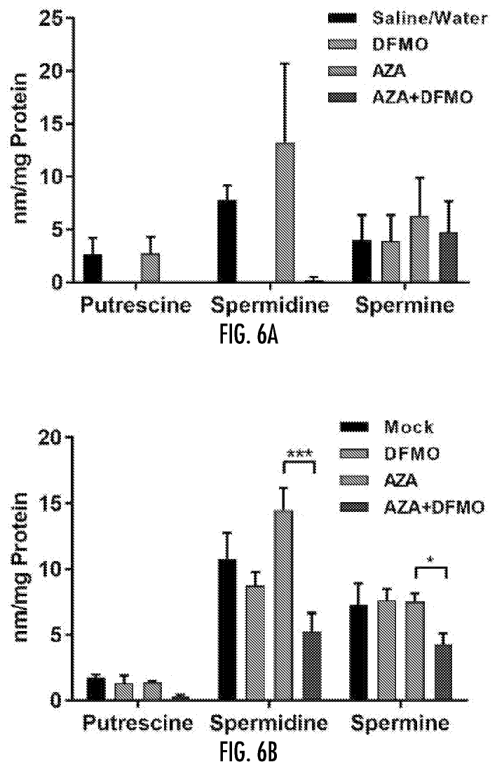

Combination AZA and DFMO therapy reduces tumor burden and increases survival in an ovarian cancer mouse model. To confirm that DFMO inhibits ODC in the model systems used, VDID8 cells were treated in vitro and in vivo and polyamine levels were determined (FIG. 6A-6B). In vitro treatment of VDID8 tumor cells led to a significant decrease in putrescine and spermidine with DFMO alone and when combined with AZA. However, AZA alone appeared to have a stimulatory effect on putrescine and spermidine synthesis (FIG. 6A). In bulk ascites cells from treated animals, combination treatment led to a decrease in all three polyamines, including spermine (FIG. 6B). No significant changes to the polyamine pools were observed with AZA treatment alone, but putrescine and spermidine were decreased (although not significantly) by DFMO treatment (FIG. 6B).

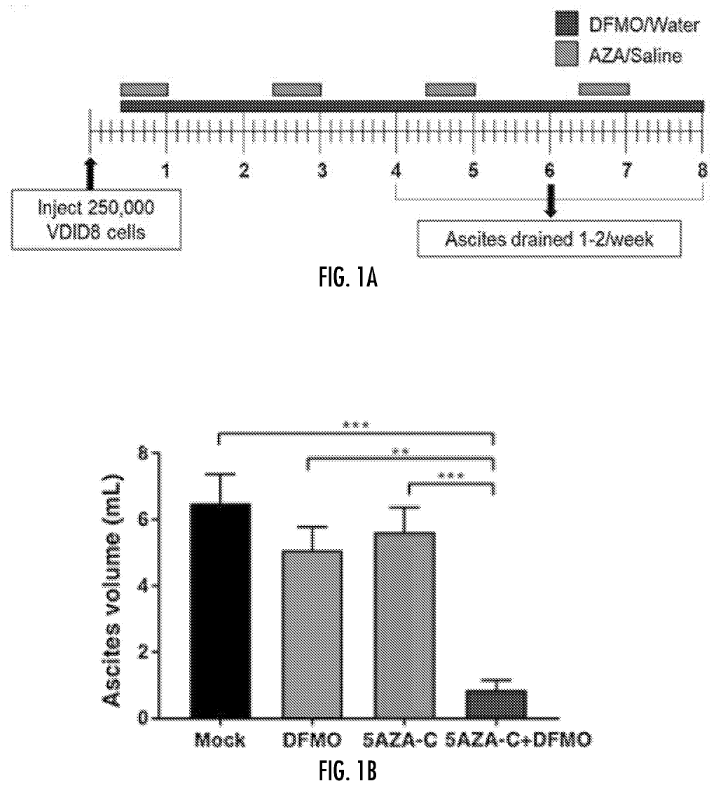

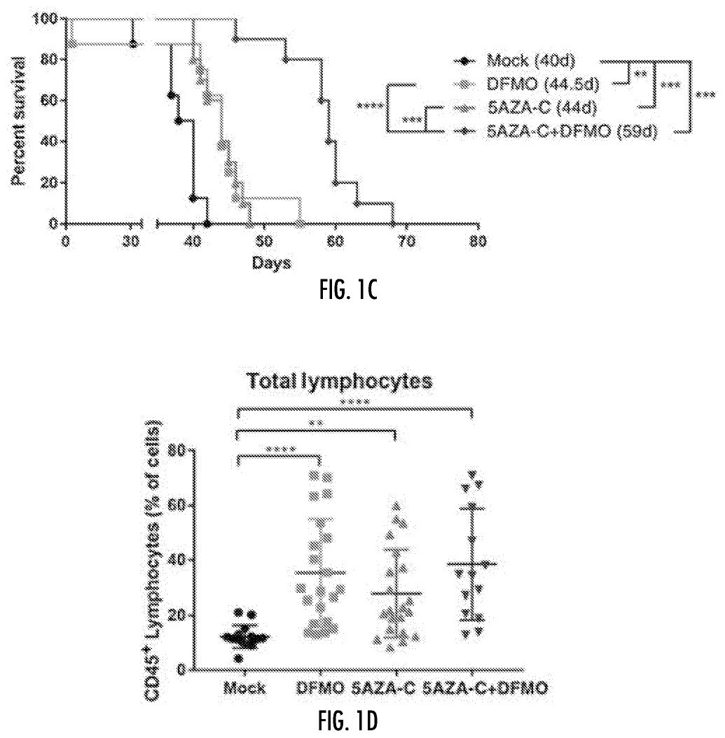

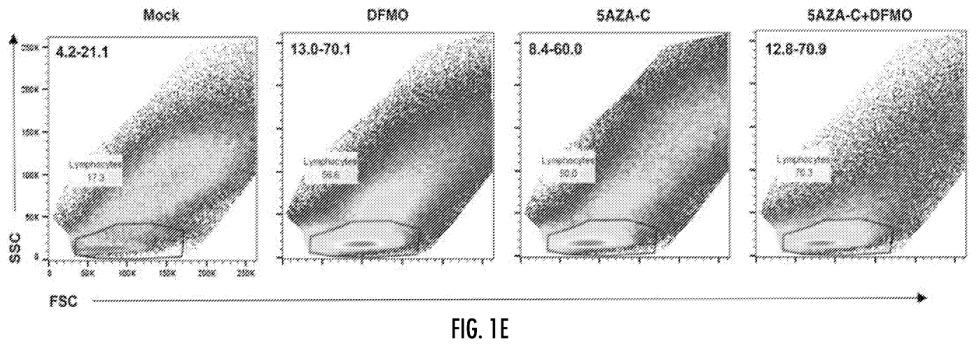

To test the hypothesis that addition of DFMO to therapy using the DNMTi AZA would reduce tumor burden and improve overall survival in a mouse model of ovarian cancer, immunocompetent C57BL/6 mice were injected intraperitoneally (i.p.) with 250,000 VDID8 syngeneic MOSE cells. Mice were treated IP with AZA (0.5 mg/kg) or saline vehicle, DFMO (2% in water), or combination AZA and DFMO beginning three days post tumor injection (FIG. 1A). Hemorrhagic ascites fluid consistently develops at approximately 4-5 weeks post VDID8 injection and is an accurate measurement of tumor burden in mice, allowing observation of tumor growth in real time (35,38). After draining hemorrhagic ascites fluid from mice for the second time (typically week 5 post tumor injection), mice treated with single agent AZA or DFMO present with higher tumor burden than mice treated with combination therapy (FIG. 1B). Mice treated with combination therapy also exhibited the largest increase in overall survival with a median survival of 59 days compared to that of single agent AZA or DFMO of approximately 44 days (FIG. 1C). Although total numbers of lymphocytes are significantly increased by single agent AZA or DFMO compared to vehicle, these numbers are not further enhanced with combination AZA+DFMO treatment (FIG. 1E-1E).

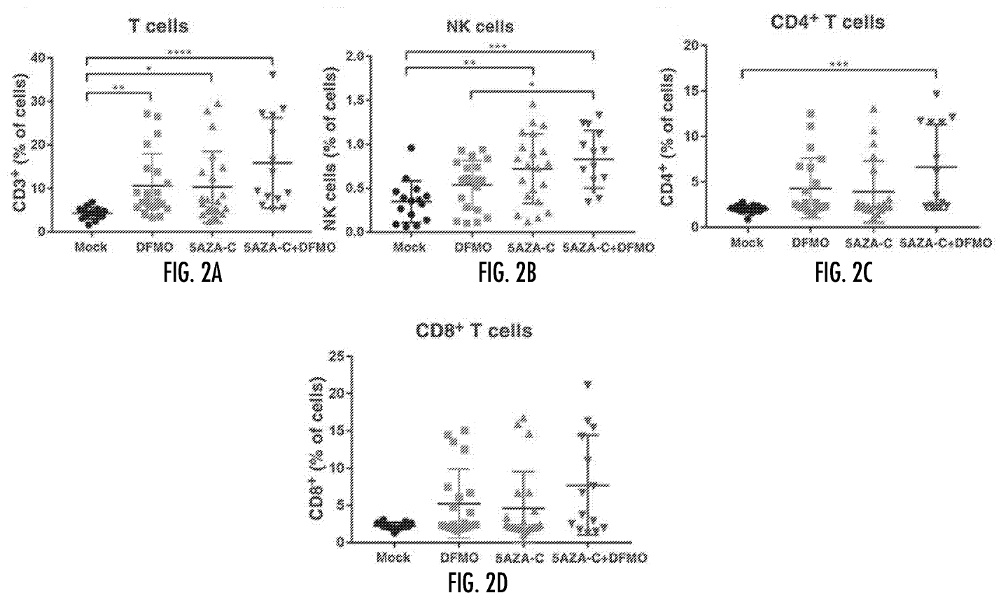

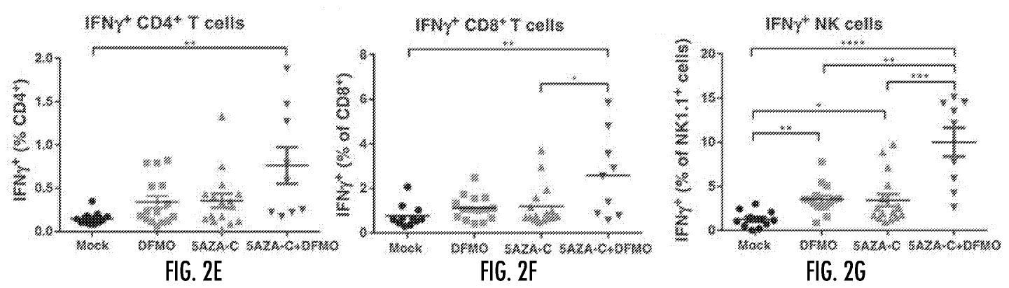

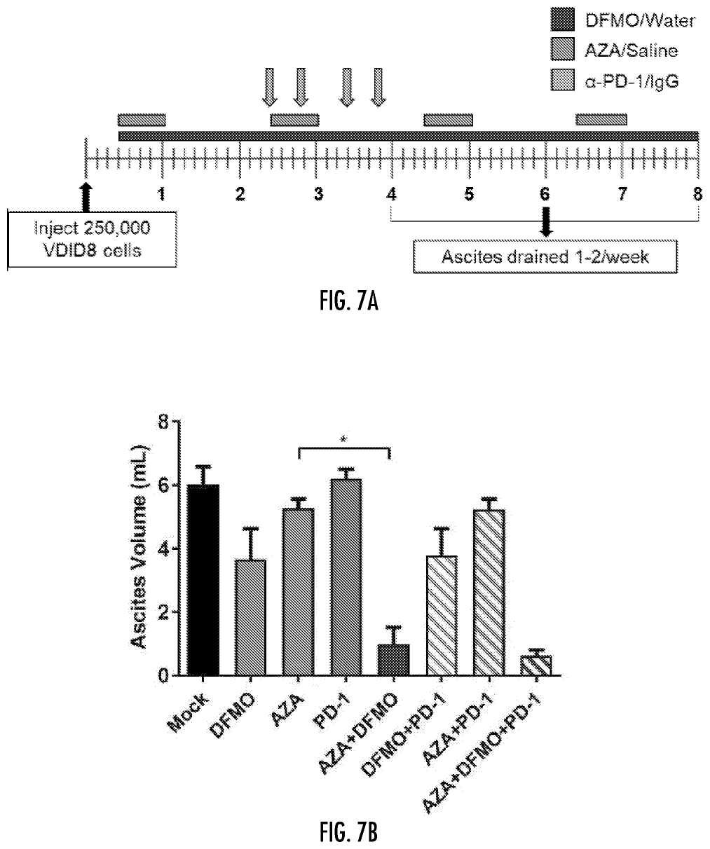

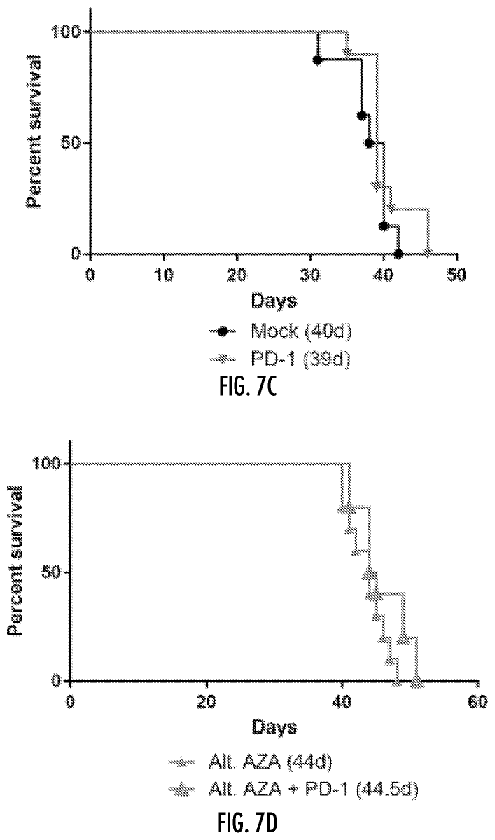

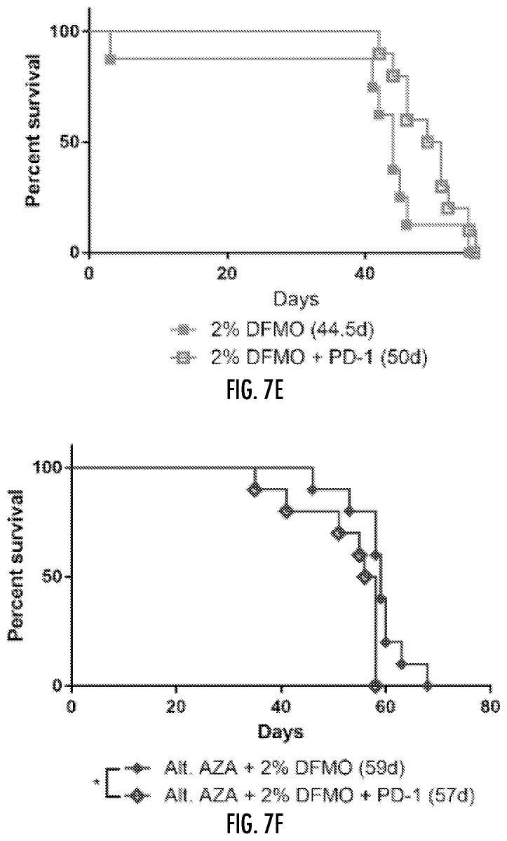

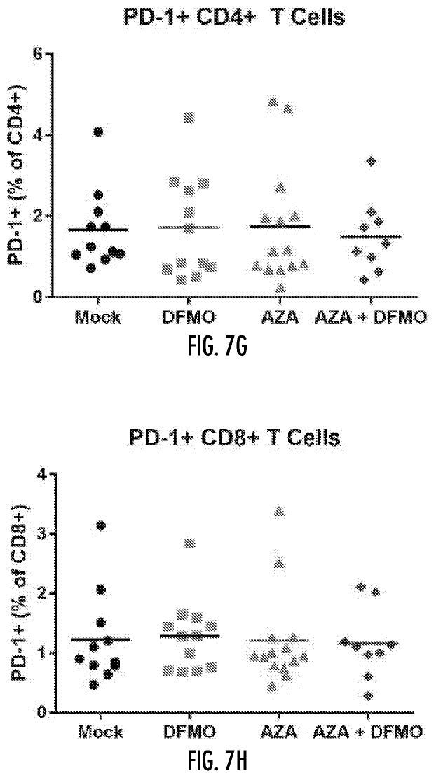

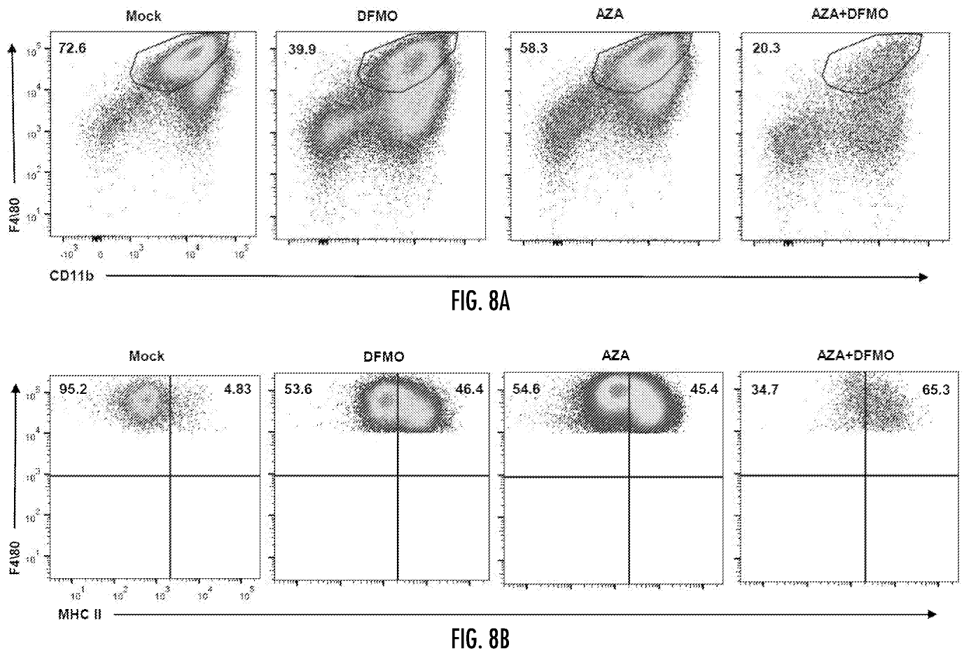

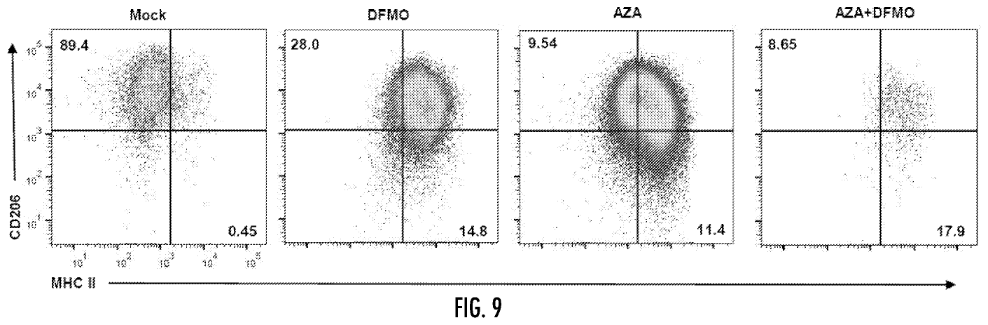

AZA and DFMO combination treatment significantly increases IFNγ+ NK cells. To pursue further whether changes in lymphocyte populations might account for the dramatic increase in survival observed with AZA+DFMO combination therapy, the numbers and activity of specific lymphocyte subpopulations in hemorrhagic ascites fluid at week 5 post tumor injection were analyzed. Single agent AZA or DFMO led to significant increases in T cell, NK cell, and IFNγ+ lymphocyte populations examined in the tumor microenvironment (FIG. 2A-2G). In most cases, combination therapy did not alter immune populations over what was observed with single agents (FIG. 2A-2F). The exception however, was a significant increase in IFNγ+ NK cells observed in combination treated mice versus AZA or DFMO alone (FIG. 2G). It was hypothesized that the observed increase in IFNγ+ cells in the model could lead to an increase in PD-L1 expression on the surface of tumor cells, possibly sensitizing the tumor to α-PD-1 therapy. Surface PD-1 expression on T cells is a signature of immune tolerance, and when engaged with its ligand PD-L1 on tumor cells, can limit the T cell's ability to proliferate and perform its effector functions (39,40). Addition of α-PD-1 to the combination of DFMO and AZA treatment did not further decrease tumor burden in the mice, nor did it increase survival (FIG. 7A-7F). No changes were observed in the number of PD-1 expressing cells with single agent or combination treatment on either CD4+ or CD8+ T cells (FIG. 2G-2H). The lack of response to α-PD-1 therapy suggests that a T cell response may not be the primary mechanism of action in this combination drug therapy. Although AZA and DFMO treatment led to elevated IFNγ+ NK cells and modest increases in T cells, it does not appear that the differences between combination treatment and single agents AZA or DFMO were significant enough to explain the dramatic increase in survival seen with combination treatment (FIG. 1C).

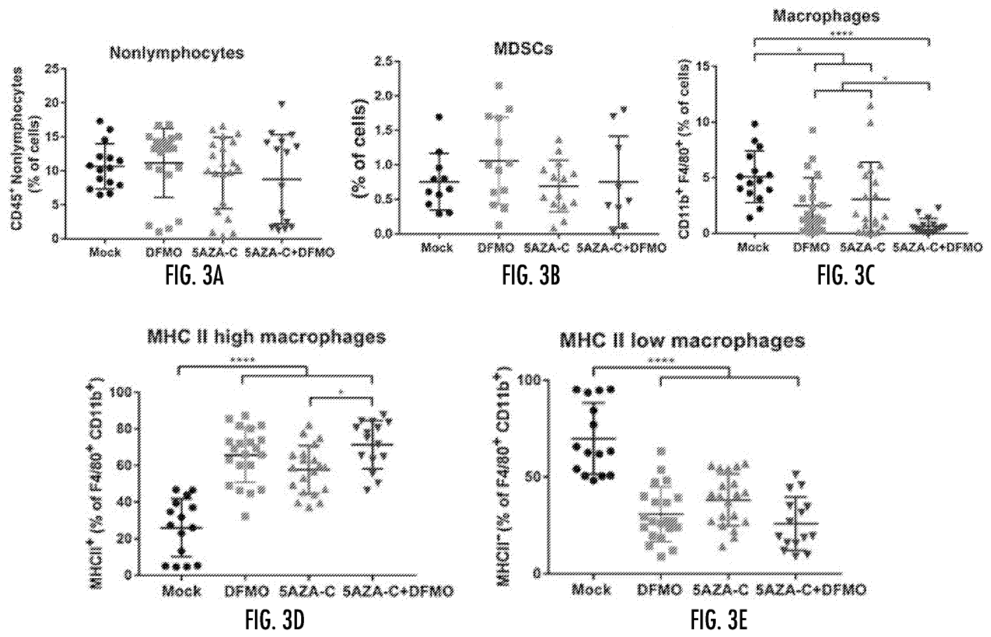

Combination AZA+DFMO treatment results in a significant decrease in macrophages. The myeloid immune cell populations were next examined to determine whether a decrease in immunosuppression may account for the striking differences in survival. MDSCs are suppressive immune cells sometimes present in the tumor microenvironment, high levels of which are associated with a poor prognosis in ovarian cancer (7). No significant decrease in non-lymphocyte or MDSC populations was observed after treatment with AZA and DFMO (FIG. 3A-3B). Instead, total macrophage populations in the tumor microenvironment were consistently decreased with AZA treatment, and decreased even further with the addition of DFMO (FIG. 3C). Macrophages are professional antigen presenting cells capable of activating T cells. Surface expression of MHC II is essential for interaction with T cells, and the number of MHC II positive cells was increased with AZA, DFMO, and AZA+DFMO treatment compared to vehicle (FIG. 3D-3E, 8A-8B). Importantly, MHC II expressing cells were increased significantly with combination treatment compared to single agent AZA, suggesting a possible explanation for the dramatic increase in survival (FIG. 3D, 1C). In contrast, untreated mice had high populations of macrophages negative for the MHC II surface protein. These data suggest that macrophages may play an important role in tumor response to the combination drug therapy.

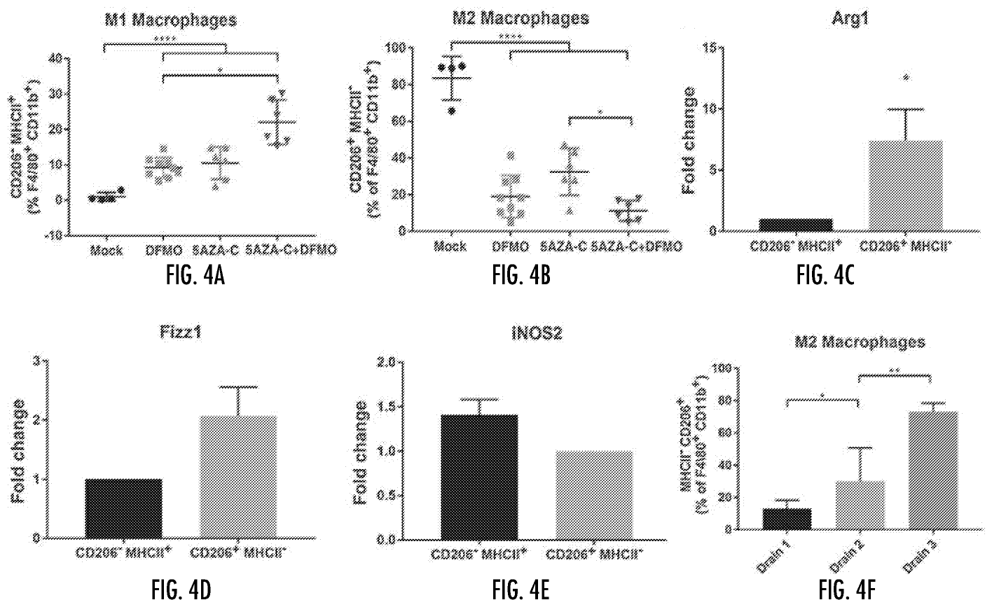

Combination AZA plus DFMO treatment leads to an increased ratio of M1 macrophages to M2 macrophages in the tumor microenvironment. Next, surface markers were examined to distinguish between classical (M1) and alternative (M2) polarized macrophages. High populations of M2 macrophages are associated with a poor prognosis due to their ability to promote tumor growth (8-10). Because the surface marker CD206 is unregulated on M2 macrophages, flow cytometry was used to analyze macrophages high for CD206 and low for MHC II—a surface marker for M1 macrophages. Although total macrophages were decreased by the treatments, an increase in M1 macrophages was observed in the remaining macrophage population for all treatment groups (FIG. 4A), as well as a decrease in M2 macrophages (FIG. 4B, 9). MHC II− CD206+ and MHC II+ CD206− macrophages were then sorted via flow cytometry, and RNA was isolated to perform RT-PCR on M1- and M2-specific genes (41-44). As expected, CD206+ macrophages demonstrated increased expression of Arg1 and Fizz1 compared to CD206− macrophages (FIG. 4C-4D), and MHC II+ macrophages had increased expression of iNOS2 compared to MHC II− macrophages (FIG. 4E). These data confirm that macrophages expressing high levels of CD206 in our model also retain gene expression patterns that are characteristic of alternatively polarized M2 macrophages.

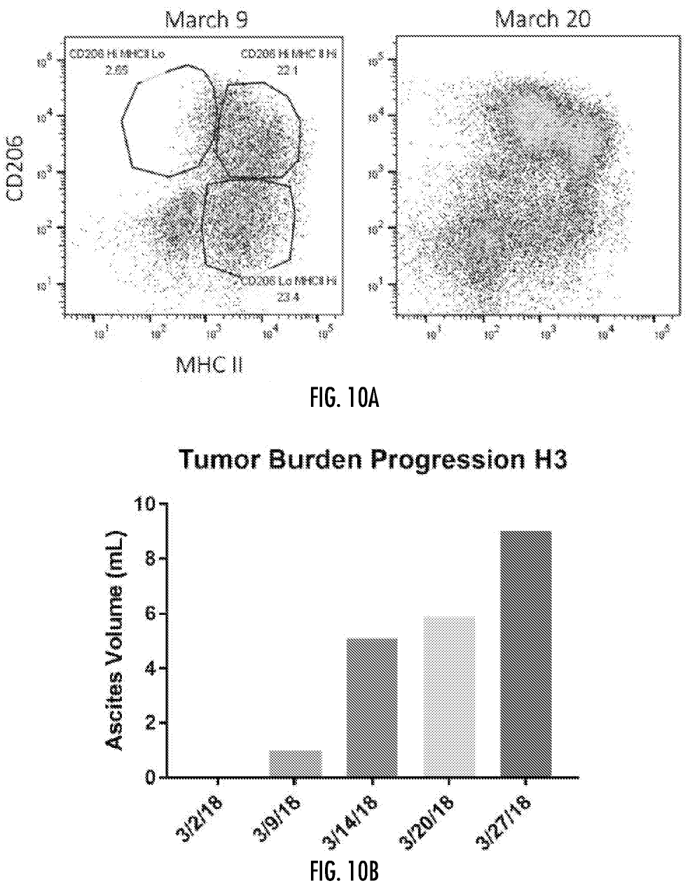

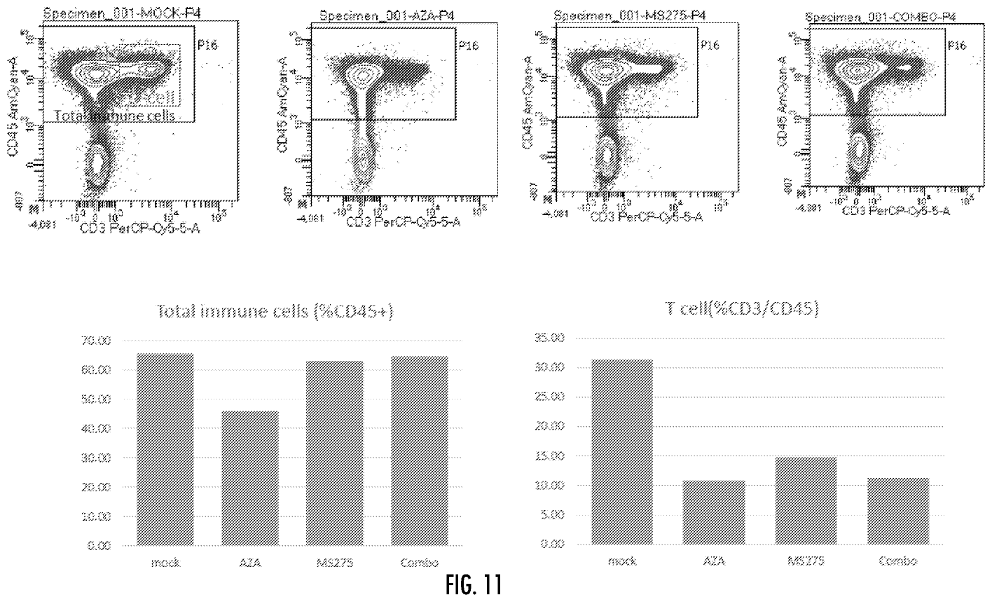

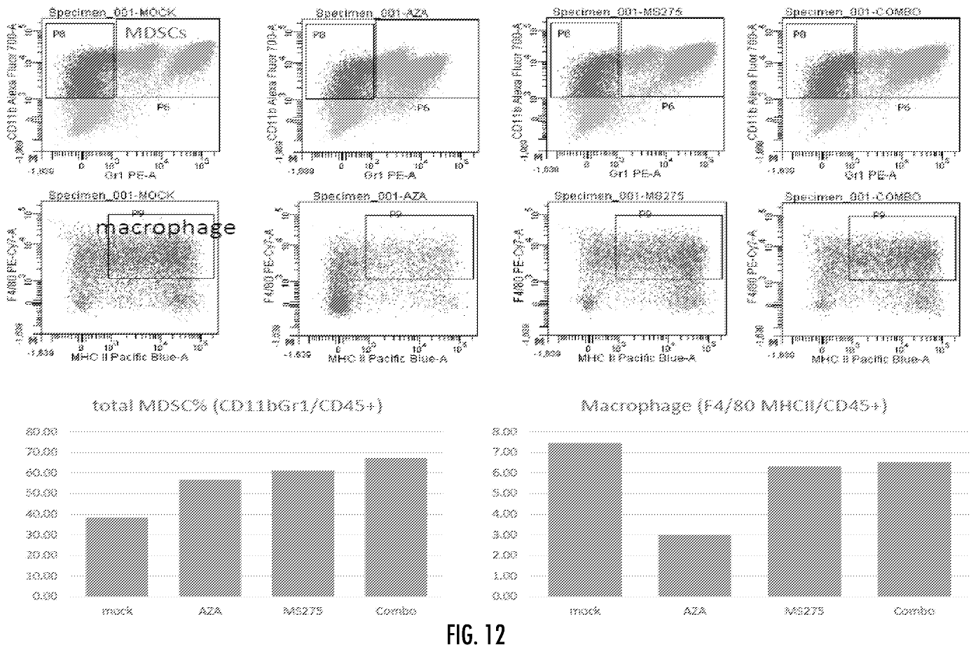

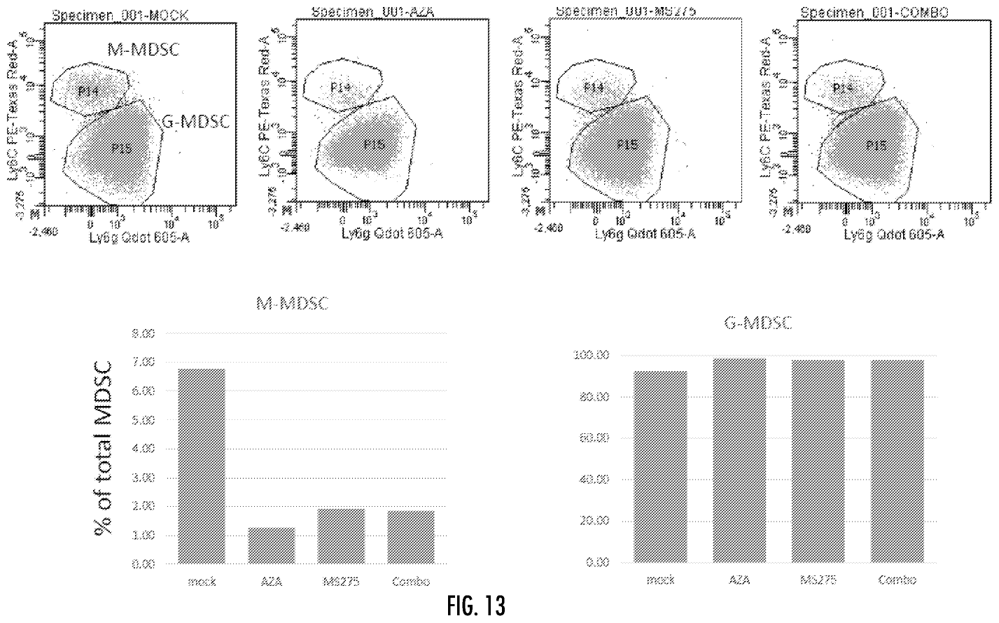

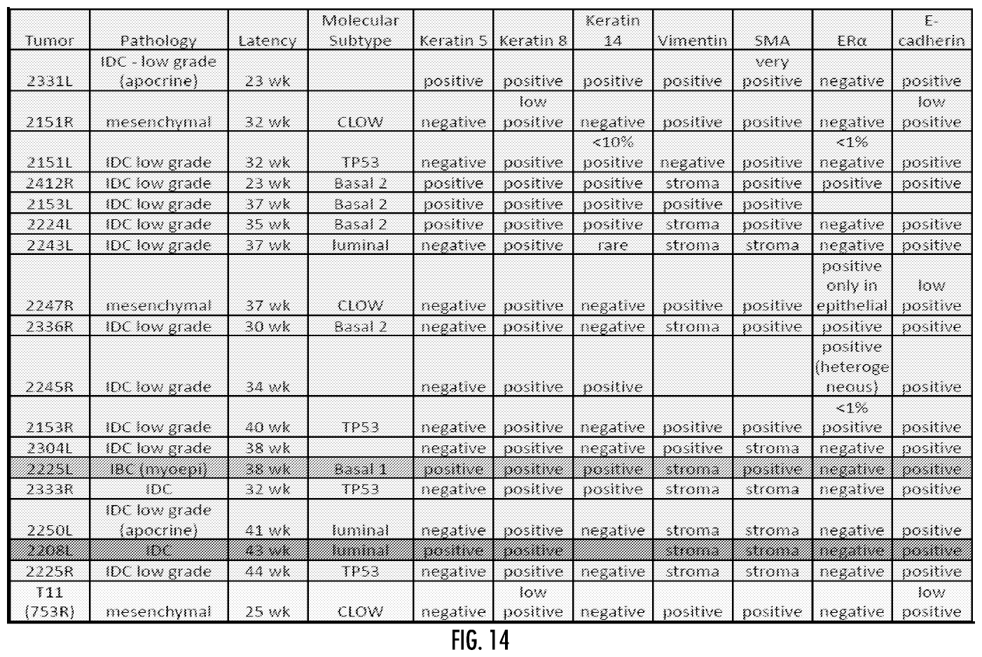

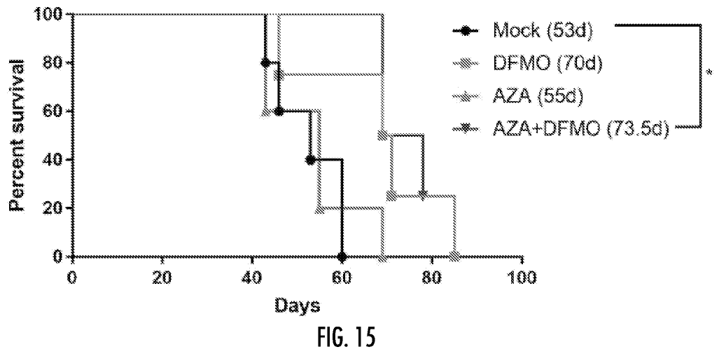

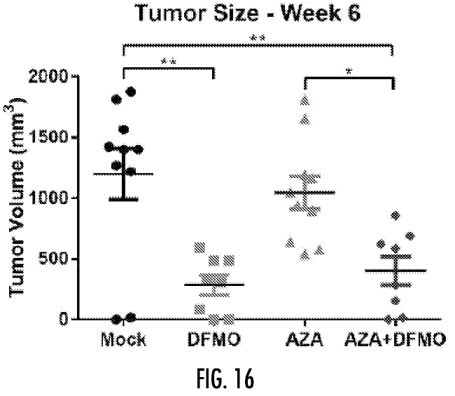

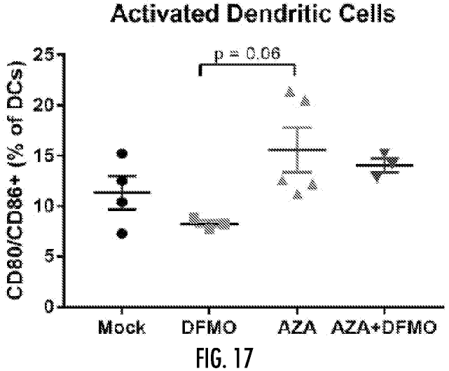

Interestingly, the decrease in M2 macrophages observed in AZA+DFMO treated mice was not a durable response, and as tumor burden increased in these mice, the relative proportion of M2 macrophages increased as well (FIG. 10A-10B). Macrophages in vehicle treated mice were therefore assessed at three different time points to determine whether M2 macrophages increase as the disease progresses. Indeed, relative levels of M2 macrophages increased as tumor burden increased in these mice, suggesting the importance of macrophages in disease progression of this ovarian cancer model (FIG. 4F).

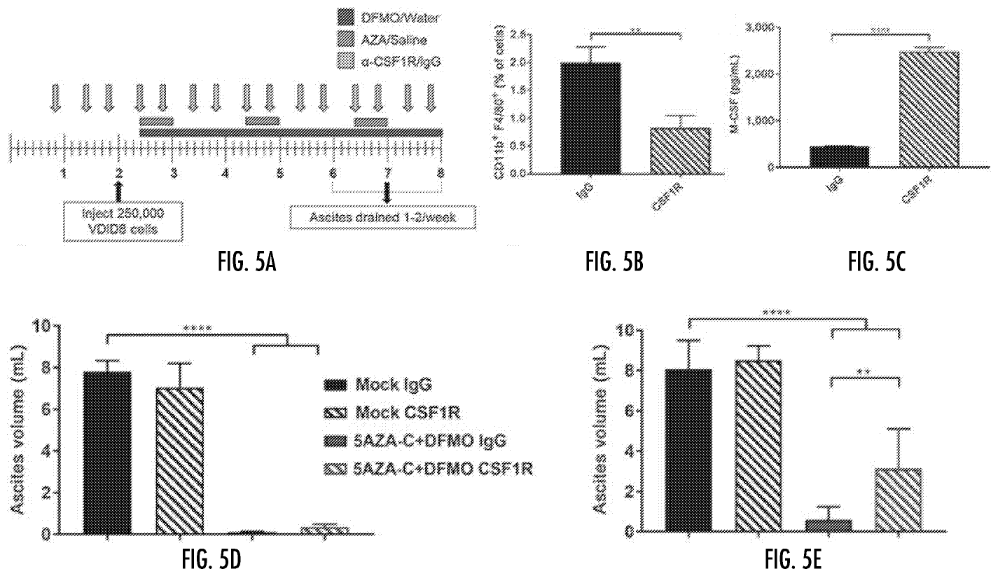

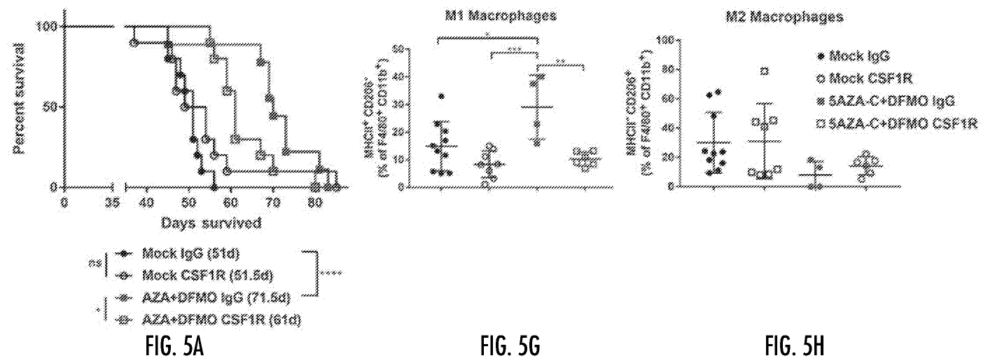

Blocking macrophages with CSF1R antibody diminishes the AZA plus DFMO response in the ovarian cancer mouse model. To test whether the increase in M1 macrophages was important in the response to AZA and DFMO treatment, macrophages were blocked in the ovarian cancer mouse model using an antibody to CSF1R (45) (FIG. 5A). Treatment with α-CSF1R resulted in decreased macrophages in the tumor microenvironment (FIG. 5B) and a consequential increase in M-CSF levels in ascites fluid as measured by ELISA (FIG. 5C). Increased M-CSF indicates that the α-CSF1 receptor block antibody is functional, as more ligand (M-CSF) is free, and less ligand is engaged with its receptor (45). Initially, the AZA+DFMO combination treatment still resulted in decreased tumor burden in mice, even with the observed decrease in macrophages; however, over time, tumor burden increased more rapidly in AZA+DFMO mice receiving α-CSF1R (FIG. 5D-5E). This decrease in macrophages also led to a decrease in overall survival, compared with AZA+DFMO mice that received IgG control (FIG. 5F). Analysis via flow cytometry of M1 and M2 surface markers showed that with IgG control, AZA+DFMO mice had increased M1 macrophages and decreased M2 macrophages compared to vehicle, as was previously seen (FIG. 5G-5H; FIG. 4A-4B). Interestingly, while AZA+DFMO mice maintained low M2 macrophages in the presence of α-CSF1R (consistent with the action of α-CSF1R, FIG. 5B), M1 macrophages were significantly decreased compared to AZA+DFMO mice receiving IgG control (FIG. 5G-5H). These results indicate that the presence of M1 macrophages is important for the mechanism of action of this combination drug therapy, as AZA+DFMO treated mice receiving α-CSF1R had decreased survival and increased tumor burden compared to IgG control.

### Discussion

Combination epigenetic and polyamine reducing therapy is an effective treatment strategy for ovarian cancer in immunocompetent mice, prolonging survival and decreasing tumor burden significantly. This treatment regimen represents the first combination of these two drug therapies in mice, and the first use of DFMO in an immunocompetent mouse model for ovarian cancer (46). Treatment with AZA alone led to an increase in IFNγ+ NK cells, CD4+ T cells, and CD8+ T cells, as has been demonstrated before (14,18,19). Signaling of IFNγ via its receptor IFNGR1 on tumor cells can lead to increased expression of PD-L1 on tumor cells, thereby making this increase in IFNγ an attractive candidate for α-PD-1 therapy. However, α-PD-1 therapy had no significant impact on survival in this model when added to the combination AZA and DFMO. These results are in contrast to previous studies using AZA and HDACi where the addition of α-PD-1 produced a significant therapeutic response (18). Histone acetylation is essential for transcription of IFNγ, therefore the use of an HDACi may explain the sensitization to α-PD-1 therapy previously seen, as increasing histone acetylation even further increased IFNγ levels in lymphocytes (47).

Analysis of the tumor microenvironment after the combination treatment with AZA and DFMO indicated that the impacts on macrophage polarization are critically important in this model. AZA treatment has been shown to decrease macrophages in the tumor microenvironment, though previously no distinction was made as to the polarization status of these macrophages (18,19). As the understanding of macrophages deepens, research has discovered that these cells once thought of as permanent, differentiated cells, are in fact quite plastic and able to respond to multiple signals including cytokines and chemokines that direct their behavior and alter their phenotype. Classically polarized M1 macrophages, induced by cytokines such as IFNγ and IL-12, upregulate expression of MHC II and can have tumoricidal functions. M1 macrophages metabolize arginine via iNOS to nitric oxide (NO), creating an oxidizing environment that is damaging to surrounding cells. DFMO treatment has been found to potentiate NO production in LPS-stimulated macrophages in vitro (48). Additionally, DFMO, via product inhibition through the increase in ODC substrate, ornithine, inhibits the enzyme arginase I, which is essential for function of alternatively polarized M2 macrophages (23,41). Inhibition of arginase I could lead to increased amounts of its substrate arginine, potentially providing more of the metabolite for use by M1 macrophages and iNOS (41,49). Treatment with DFMO may therefore increase M1 macrophages by making more of its essential metabolite arginine available, while AZA may help increase M1 macrophages via its interferon response and production of IFNγ, a cytokine which drives M1 polarization (15,18,19,49).

Depletion of macrophages in the tumor microenvironment using a CSF1R antibody significantly diminished the efficacy of combination AZA and DFMO, and decreased the levels of M1 macrophages. Tumor burden recurred more rapidly and survival was diminished in mice with fewer macrophages, suggesting that these M1 macrophages could have a tumoricidal role in ovarian tumors. This work represents the first combination of these two distinct treatment strategies in any cancer. The impact of AZA and DFMO on macrophages in the tumor microenvironment may not be specific to ovarian cancer, and could therefore possibly translate to other macrophage-rich tumors. Furthermore, the use of two well-tolerated and clinically approved drugs offers potential to test a third drug in combination to further prolong survival. Exploration of additional drugs that potentiate M1 macrophages is important, as these tumoricidal cells have potential to decrease tumor burden and help activate the immune system against cancer.

### REFERENCES

1. Siegel R L, Miller K D, Jemal A. Cancer statistics, 2018. CA Cancer J Clin 2018;68:7-30.

2. Alipour S, Zoghi S, Khalili N, Hirbod-Mobarakeh A, Emens L A, Rezaei N. Specific immunotherapy in ovarian cancer: a systematic review. Immunotherapy 2016;8:1193-204.

3. Chester C, Dorigo O, Berek JS, Kohrt H. Immunotherapeutic approaches to ovarian cancer treatment. J Immunother Cancer 2015;3:7.

4. Preston C C, Goode E L, Hartmann L C, Kalli K R, Knutson K L. Immunity and immune suppression in human ovarian cancer. Immunotherapy 2011;3:539-56.

5. Brahmer J R, Tykodi S S, Chow L Q, Hwu W J, Topalian S L, Hwu P, et al. Safety and activity of anti-PD-L1 antibody in patients with advanced cancer. N Engl J Med 2012;366:2455-65.

6. Leffers N, Gooden M J, de Jong R A, Hoogeboom B N, ten Hoor K A, Hollema H, et al. Prognostic significance of tumor-infiltrating T-lymphocytes in primary and metastatic lesions of advanced stage ovarian cancer. Cancer Immunol Immunother 2009;58:449-59.

7. Wu L, Deng Z, Peng Y, Han L, Liu J, Wang L, et al. Ascites-derived IL-6 and IL-10 synergistically expand CD14(+)HLA-DR(-/low) myeloid-derived suppressor cells in ovarian cancer patients. Oncotarget 2017;8:76843-56.

8. Zhang M, He Y, Sun X, Li Q, Wang W, Zhao A, et al. A high M1/M2 ratio of tumor-associated macrophages is associated with extended survival in ovarian cancer patients. J Ovarian Res 2014;7:19.

9. Yuan X, Zhang J, Li D, Mao Y, Mo F, Du W, et al. Prognostic significance of tumor-associated macrophages in ovarian cancer: A meta-analysis. Gynecol Oncol 2017;147:181-7.

10. Galli S J, Borregaard N, Wynn T A. Phenotypic and functional plasticity of cells of innate immunity: macrophages, mast cells and neutrophils. Nat Immunol 2011;12:1035-44.

11. Bingle L, Brown N J, Lewis C E. The role of tumour-associated macrophages in tumour progression: implications for new anticancer therapies. J Pathol 2002;196:254-65.

12. Tamura R, Tanaka T, Yamamoto Y, Akasaki Y, Sasaki H. Dual role of macrophage in tumor immunity. Immunotherapy 2018;10:899-909.

13. Wrangle J, Wang W, Koch A, Easwaran H, Mohammad HP, Vendetti F, et al. Alterations of immune response of Non-Small Cell Lung Cancer with Azacytidine. Oncotarget 2013;4:2067-79.

14. Li H, Chiappinelli K B, Guzzetta A A, Easwaran H, Yen R W, Vatapalli R, et al. Immune regulation by low doses of the DNA methyltransferase inhibitor 5-azacitidine in common human epithelial cancers. Oncotarget 2014;5:587-98.

15. Chiappinelli K B, Strissel P L, Desrichard A, Li H, Henke C, Akman B, et al. Inhibiting DNA Methylation Causes an Interferon Response in Cancer via dsRNA Including Endogenous Retroviruses. Cell 2015;162:974-86.

16. Roulois D, Loo Yau H, Singhania R, Wang Y, Danesh A, Shen S Y, et al. DNA-Demethylating Agents Target Colorectal Cancer Cells by Inducing Viral Mimicry by Endogenous Transcripts. Cell 2015;162:961-73.

17. Wang L, Amoozgar Z, Huang J, Saleh M H, Xing D, Orsulic S, et al. Decitabine Enhances Lymphocyte Migration and Function and Synergizes with CTLA-4 Blockade in a Murine Ovarian Cancer Model. Cancer Immunol Res 2015;3:1030-41.

18. Stone M L, Chiappinelli K B, Li H, Murphy L M, Travers M E, Topper M J, et al. Epigenetic therapy activates type I interferon signaling in murine ovarian cancer to reduce immunosuppression and tumor burden. Proc Natl Acad Sci USA 2017;114:E10981-E90.

19. Topper M J, Vaz M, Chiappinelli K B, DeStefano Shields C E, Niknafs N, Yen R C, et al. Epigenetic Therapy Ties MYC Depletion to Reversing Immune Evasion and Treating Lung Cancer. Cell 2017;171:1284-300 e21.

20. Tsai H C, Li H, Van Neste L, Cai Y, Robert C, Rassool F V, et al. Transient low doses of DNA-demethylating agents exert durable antitumor effects on hematological and epithelial tumor cells. Cancer Cell 2012;21:430-46.

21. Juergens R A, Wrangle J, Vendetti F P, Murphy S C, Zhao M, Coleman B, et al. Combination epigenetic therapy has efficacy in patients with refractory advanced non-small cell lung cancer. Cancer Discov 2011;1:598-607.

22. Hayes C S, Shicora A C, Keough M P, Snook A E, Burns M R, Gilmour S K. Polyamine-blocking therapy reverses immunosuppression in the tumor microenvironment. Cancer Immunol Res 2014;2:274-85.

23. Casero R A, Jr., Murray Stewart T, Pegg A E. Polyamine metabolism and cancer: treatments, challenges and opportunities. Nat Rev Cancer 2018.

24. Pegg A E. Regulation of ornithine decarboxylase. J Biol Chem 2006;281:14529-32.

25. Abeloff M D, Rosen S T, Luk G D, Baylin S B, Zeltzman M, Sjoerdsma A. Phase II trials of alpha-difluoromethylornithine, an inhibitor of polyamine synthesis, in advanced small cell lung cancer and colon cancer. Cancer Treat Rep 1986;70:843-5.

26. Abeloff M D, Slavik M, Luk G D, Griffin C A, Hermann J, Blanc O, et al. Phase I trial and pharmacokinetic studies of alpha-difluoromethylornithine—an inhibitor of polyamine biosynthesis. J Clin Oncol 1984;2:124-30.

27. Bassin H, Benavides A, Haber M, Gilmour S K, Norris M D, Hogarty M D. Translational development of difluoromethylornithine (DFMO) for the treatment of neuroblastoma. Transl Pediatr 2015;4:226-38.

28. Heby O, Persson L, Rentala M. Targeting the polyamine biosynthetic enzymes: a promising approach to therapy of African sleeping sickness, Chagas' disease, and leishmaniasis. Amino Acids 2007;33:359-66.

29. Kansiime F, Adibaku S, Wamboga C, Idi F, Kato C D, Yamuah L, et al. A multicentre, randomised, non-inferiority clinical trial comparing a nifurtimox-eflornithine combination to standard eflornithine monotherapy for late stage Trypanosoma brucei gambiense human African trypanosomiasis in Uganda. Parasit Vectors 2018;11:105.

30. Laukaitis C M, Gerner E W. DFMO: targeted risk reduction therapy for colorectal neoplasia. Best Pract Res Clin Gastroenterol 2011;25:495-506.

31. Meyskens F L, Jr., McLaren C E, Pelot D, Fujikawa-Brooks S, Carpenter P M, Hawk E, et al. Difluoromethylornithine plus sulindac for the prevention of sporadic colorectal adenomas: a randomized placebo-controlled, double-blind trial. Cancer Prey Res (Phila) 2008;1:32-8.

32. Zell J A, Pelot D, Chen W P, McLaren C E, Gerner E W, Meyskens F L. Risk of cardiovascular events in a randomized placebo-controlled, double-blind trial of difluoromethylornithine plus sulindac for the prevention of sporadic colorectal adenomas. Cancer Prey Res (Phila) 2009;2:209-12.

33. Ye C, Geng Z, Dominguez D, Chen S, Fan J, Qin L, et al. Targeting Ornithine Decarboxylase by alpha-Difluoromethylornithine Inhibits Tumor Growth by Impairing Myeloid-Derived Suppressor Cells. J Immunol 2016;196:915-23.

34. Roby K F, Taylor C C, Sweetwood J P, Cheng Y, Pace J L, Tawfik O, et al. Development of a syngeneic mouse model for events related to ovarian cancer. Carcinogenesis 2000;21:585-91.

35. Conejo-Garcia J R, Benencia F, Courreges M C, Kang E, Mohamed-Hadley A, Buckanovich R J, et al. Tumor-infiltrating dendritic cell precursors recruited by a beta-defensin contribute to vasculogenesis under the influence of Vegf-A. Nat Med 2004;10:950-8.

36. Kabra P M, Lee H K, Lubich W P, Marton U. Solid-phase extraction and determination of dansyl derivatives of unconjugated and acetylated polyamines by reversed-phase liquid chromatography: improved separation systems for polyamines in cerebrospinal fluid, urine and tissue. J Chromatogr 1986;380:19-32.

37. Peirce C S. The numerical measure of the success of predictions. Science 1884;4:453-4.

38. Duraiswamy J, Freeman G J, Coukos G. Therapeutic PD-1 pathway blockade augments with other modalities of immunotherapy T-cell function to prevent immune decline in ovarian cancer. Cancer Res 2013;73:6900-12.

39. Wherry E J, Kurachi M. Molecular and cellular insights into T cell exhaustion. Nat Rev Immunol 2015;15:486-99.

40. Zehn D, Wherry E J. Immune Memory and Exhaustion: Clinically Relevant Lessons from the LCMV Model. Adv Exp Med Biol 2015;850:137-52.

41. Bronte V, Zanovello P. Regulation of immune responses by L-arginine metabolism. Nat Rev Immunol 2005;5:641-54.

42. Lawrence T, Natoli G. Transcriptional regulation of macrophage polarization: enabling diversity with identity. Nat Rev Immunol 2011;11:750-61.

43. Sica A, Mantovani A. Macrophage plasticity and polarization: in vivo veritas. J Clin Invest 2012;122:787-95.

44. Martinez F O, Gordon S. The M1 and M2 paradigm of macrophage activation: time for reassessment. F1000Prime Rep 2014;6:13.

45. MacDonald K P, Palmer J S, Cronau S, Seppanen E, Olver S, Raffelt N C, et al. An antibody against the colony-stimulating factor 1 receptor depletes the resident subset of monocytes and tissue- and tumor-associated macrophages but does not inhibit inflammation. Blood 2010;116:3955-63.

46. Manetta A, Satyaswarcoop P G, Podczaski E S, Hamilton T, Ozols R F, Mortel R. Effect of alpha-difluoromethylornithine (DFMO) on the growth of human ovarian carcinoma. Eur J Gynaecol Oncol 1988;9:222-7.

47. Peng M, Yin N, Chhangawala S, Xu K, Leslie CS , Li M O. Aerobic glycolysis promotes T helper 1 cell differentiation through an epigenetic mechanism. Science 2016;354:481-4.

48. Baydoun A R, Morgan D M. Inhibition of ornithine decarboxylase potentiates nitric oxide production in LPS-activated J774 cells. Br J Pharmacol 1998;125:1511-6.

49. Mantovani A, Marchesi F, Malesci A, Laghi L, Allavena P. Tumour-associated macrophages as treatment targets in oncology. Nat Rev Clin Oncol 2017;14:399-416.

1. A method for treating a solid tumor in a patient comprising the step of administering to the patient a polyamine reduction therapy and an epigenetic therapy.

2. The method of claim 1, wherein the polyamine reduction therapy comprises a polyamine synthesis inhibitor and/or a polyamine transport inhibitor.

3. The method of claim 2, wherein the polyamine synthesis inhibitor comprises an ornithine decarboxylase (ODC) inhibitor.

4. The method of claim 3, wherein the ODC inhibitor comprises difluoromethylornithine (DFMO).

5. The method of claim 2, wherein the polyamine transport inhibitor comprises AMXT-1501.

6. The method of claim 1, wherein the epigenetic therapy comprises a DNA methyl tranferase inhibitor (DNMTi), a histone deastylase inhibitor (HDACi), a histone methyltransferase inhibitor and/or a histone demethylase inhibitor.

7. The method of claim 6, wherein the DNMTi comprises 5-azacytidine (AZA) or 5-azadeoxycytidine (DAC).

8. The method of claim 6, wherein the HDACi inhibits class I and class II histone deacetylases.

9. The method of claim 6, wherein the HDACi comprises givinostat or entinostat.

10. The method of claim 1, further comprising administering a checkpoint inhibitor.

11. The method of claim 10, wherein the checkpoint inhibitor comprises an anti-PD1 antibody, an anti-PDL-1 antibody or an anti-CTLA4 antibody.

12. The method of claim 11, wherein the anti-PD1 antibody comprises nivolumab or pembrolizumab.

13. The method of claim 11, wherein the anti-PDL-1 antibody comprises Medi4736 or MPDL3280A.

14. The method of claim 11, wherein the anti-CTLA4 antibody comprises tremelimumab.

15. The method of claim 1, wherein the patient is administered an epigenetic therapy prior to the step of administering the polyamine reduction therapy and the epigenetic therapy.

16. The method of claim 1, further comprising administering an agent that reduces M2 macrophages.

17. The method of claim 16, wherein the agent is and anti-interleukin-10 receptor (IL-10R) antibody.

18. A method for treating a solid tumor in a patient comprising the step of administering:
(a) a polyamine reduction therapy comprising either (i) DFMO or (ii) AMXT-1501;
(b) an epigenetic therapy comprising either (i) AZA or DAC or (ii) givinostat or entinostat.

19. The method of claim 18, further comprising (c) a checkpoint inhibitor comprising nivolumab, pembrolizumab, Medi4736, MPDL3280A or tremelimumab.

20. The method of claim 18, further comprising (d) an agent that reduces M2 macrophage comprising anti-IL10R antibody.

21. A pharmaceutical composition comprising (a) either an ODC inhibitor or a polyamine transport inhibitor; and (b) either a DNMTi or a HDACi.

22. The pharmaceutical composition of claim 21, wherein the ODC inhibitor comprises DFMO.

23. The pharmaceutical composition of claim 21, wherein the polyamine transport inhibitor comprises AMXT-1501.

24. The pharmaceutical composition of claim 21, wherein the DNMTi comprises AZA or DAC.

25. The pharmaceutical composition of claim 21, wherein the HDACi comprises givinostat or entinostat.

26. The pharmaceutical composition of claim 21, further comprising (c) a checkpoint inhibitor.

27. The pharmaceutical composition of claim 26, wherein the checkpoint inhibitor comprises nivolumab, pembrolizumab Medi4736 or tremelimumab.

28. The pharmaceutical composition of claim 21, further comprising an agent that reduces M2 macrophages.

29. The pharmaceutical composition of claim 28, wherein the agent comprises an anti-IL-10R antibody.

30. A kit comprising a polyamine reduction therapy and an epigenetic therapy.

31. The kit of claim 30, wherein the polyamine reduction therapy comprises a polyamine synthesis inhibitor or a polyamine transport inhibitor.

32. The kit of claim 30, wherein the epigenetic therapy comprises a DNMTi, an HDACi a histone methyltransferase inhibitor and/or a histone demethylase inhibitor.

33. The kit of claim 30, further comprising a checkpoint inhibitor.

34. The kit of claim 30, further comprising an agent that reduces M2 macrophages.

35. The kit of claim 30, further comprising instructions for administration to patients to treat solid tumors.
